SEQUENCE LISTING (12 sequences)

SEQ ID NO: 1 - DNA, 2850 bp, Homo sapiens
aagccctgaagggtcaaaagaaatacaaaagcaaaggctattttctttttttttttctttctttcattcmttccttcctctgtttctttctttcttcctttcatttttttttcttttttaagagcgagcggctctgcggtggcggtttggggtgggcgccgccgaggtgaggtcgtctcgcctcccgcgcgccggtagattggttgtttcattatggatggaggggatgatggtaaccttattatcaaaaagaggtttgtgtctgaggcagaactagatgaacggcgcaaaaggaggcaagaagaatgggagaaagttcgaaaacctgaagatccagaagaatgtccagaggaggtttatgaccctcgatctctatatgaaaggctacaggaacagaaggacaggaagcagcaggagtacgaggaacagttcaaattcaaaaacatggtaagaggcttagatgaagatgagaccaacttccttgatgaggtttctcgacagcaggaactaatagaaaagcaacgaagagaagaagaactgaaagaactgaaggaatacagaaataacctcaagaaggttggaatttctcaagagaacaagaaggaagtggaaaagaaactgactgtgaagcctatagaaaccaagaacaagttctcccaggcgaagctgttggcaggagctgtgaagcataagagctcagagagtggcaacagtgtgaaaagactgaaaccggaccctgagccagatgacaagaatcaagagccctcatcctgcaagtctctcggaaacacctccctgagtggcccctccatccactgcccctctgctgcagtatgtatcggcatcctcccaggcctgggtgcctactctgggagcagcgactccgagtccagctcagacagcgaaggcaccatcaatgccaccggaaagattgtctcctccatcttccgaaccaacaccttcctcgaggccccctagtttctccgtccctacacagggagctcctccccaagggtagatcggaccgttcatgctgcctataggcattatgtccctcaaaaaaaaactcctttgcctgcatcctgtgtacaacatgacatttttaaccaatccaatctaaaaatgtgccagaatccacctgtggcccgaatcgtgtttggttcctctttctactccactgcagatgaccaaacctgtcccgctgccactttcctcactgatattgggaggagggcaaggcccagccgaagttccactaaaaatgccccaggagaataggcaccggctggcttgccaaagggtttgggttttattgctttctgttttttcttttcccgacagcacaaagaagtaagggcagttattggacaggtgttatttaaacattctattgtaaatgaatgtgttgtttggttctactgcattgtggagcatgcgggggaagagaactgacccaggtaatgaaatggagcccttccctggaactaaccagtccttgatgttgtgtgactaagtaaagatgataaaccccatctgctgggggtgtcacttcacactcggcatgcattgtgaaagctttccatacccttggccattccctctctcctctctctccaaccccatttatgcaggaagggactgctaacaagaacgcttccatctcaaaccttttctctgcctgggaaattattttatgtttgtttttgaaataaaggatttagtttaagattctaaattttagagaaacaaacgtaggccttgtttactaatagccagacatcagaactgcaggtaggtatgttaatgagatgacttatttctggcagctcctggaatcctaatattgtaaatgagtgggacacacttgcatattgtgaccattctattgaggccctctctgtttaatgcatattatacttgtgcttttaactgtggaatctatttctaacctaaaggtgctgccctagtacttttctttgctgcctctgctgctctttttcctttccaaacagcaactctgaggccatgagcagccaaaaactagaggtactgctccacctcgtctcataaagggaaacgggctcatcccttggattctggaggagggagagggagatggtgtggaggcctcgaggacagagatagacatgagctttgacaacaatctgtaggctctcctgctttagaataagcatgtaccattctttatccattccccttattcctacatcaattgtttttactttcttgggtgtgagactgagtgagacacacacaaaatgtgttgacactgtgatgccggcaggcagagcagctactgactttgaacatgggcagagaggcccctggatctcatccagcccactccttttccccttccagtacagtgacactctggtgcccattggcagatggcgacttccctgcacccataactgatgctttgtgaattcttcctccttttcagaactactctgtgctaattgttctgccagtatgggcgcatcagctccatcctgacaaacaagacatttaggtaaaactttgtaggcaccttctgcttctctgcttcattgttcctgtgatagtcctgttgttattacagcatgtacccaaaacagcctcacattgttacaggaggcaggccaggacatcaaagtcatcatctttatgtggcatgactcttaagaggccattactgtatctcatggcctcttgatgtggaaagaagttgacagagggttgcagggtttaaaaacatccattaacatgaaagctaataaacctgtcagagaacaaaaaaaaaaaaaaaaaaaaaaaaaaaaaaaaaaaaaaaaaaaaaaaaaaaaa

SEQ ID NO: 2 - protein, 254 aa, Homo sapiens
MDGGDDGNLIIKKRFVSEAELDERRKRRQEEWEKVRKPEDPEECPEEVYDPRSLYERLQEQKDRKQQEYEEQFKFKNMVRGLDEDETNFLDEVSRQQELIEKQRREEELKELKEYRNNLKKVGISQENKKEVEKKLTVKPIETKNKFSQAKLLAGAVKHKSSESGNSVKRLKPDPEPDDKNQEPSSCKSLGNTSLSGPSIHCPSAAVCIGILPGLGAYSGSSDSESSSDSEGTINATGKIVSSIFRTNTFLEAP

SEQ ID NO: 3 - DNA, 1516 bp, Homo sapiens
cgtccggagcctgggggaaaagcggcgcgggagccggcacccaccgctggaggggcggcgacggcggccgtagcgacctcgggaggcaagcggagccgccatggccgagttcccgtcgaaagttagcacgcggaccagcagtcctgcgcagggcgccgaagcctcggtgtcggcgctgcgcccggacctgggcttcgtgcgctcccgcctcggggcgctcatgctgctgcagctggtgctggggctgctggtgtgggcgctgattgcggacaccccgtaccacctgtatccggcctatggctgggtgatgttcgtcgctgtcttcctctggctggtgacaatcgtcctcttcaacctctacctgtttcagctgcacatgaagttgtacatggttccctggccactggtgttaatgatctttaacatcagcgccaccgttctctacatcaccgccttcatcgcctgctctgcggcagttgacctgacatccctgaggggcacccggccttataaccagcgcgcggctgcctcgttctttgcgtgtttggtgatgatcgcctatggagtgagtgccttcttcagctaccaggcctggcgaggagtaggcagcaatgcggccaccagtcagatggctggcggctatgcctaaaccacctgtgccacggccccctctggggctgaagccgccgctgggtcacagagcagggtcaccctgcaagcctgaagctggggagccctgcgtggagtcagcccaacagggactgcatttgctcctctctgcccgtcagacataagctctcacagcgctaaggaagcaggcccaggctggcaggcatctcggcttgcaggaggccaactgctgagacctcttctccatcccccttattcagtggaagatgacgggggatctgaggctgtgtctctgccttgtctttagaggacttcagcgtccaagactggggcccacccttctcaccagcactaaatgcactaacaaggactccagacctgcagccccagacccgccgtagtataagcctaacaagcaacacgtagcaccttagtctttgttccaggagagctgagcaagctggtgaaaccactctccttcctttaaacaccgtttcaaccaacctctccctggagccaacctgtaaaaagtgggttgattgctgacagcatggtcttccctccctgcatttcagacataccagttactgaaagcaaatcagttttaagtgatttctcagtgctgaaaagcctgtccaggtttccttccctttcccaagcctctctctgtaatactccctttgggcgaagctaacatcggtgcctccccgaccttgctgactaggcacatgggacgcaaaggagggagggaagcaaggccttgcctggcgagttgtcatgtggttggtggtgactgttttattttttttaataaaaataaagatgagagaaattaaaaaaaaaaaaaaaaaaaaaaaaaaaaaaaaaaaaaaaaaaaaaaaaaaaa

SEQ ID NO: 4 - protein, 216 aa, Homo sapiens
MAEFPSKVSTRTSSPAQGAEASVSALRPDLGFVRSRLGALMLLQLVLGLLVWALIADTPYHLYPAYGWVMFVAVFLWLVTIVLFNLYLFQLHMKLYMVPWPLVLMIFNISATVLYITAFIACSAAVDLTSLRGTRPYNQRAAASFFACLVMIAYGVSAFFSYQAWRGVGSNAATSQMAGGYATTCATAPSGAEAAAGSQSRVTLQASWGALRGVSP

SEQ ID NO: 5 - DNA, 2177 bp, Homo sapiens
gtgctgtgaggggcttcgggaccttggggcagctcctgagttcagacagagttcaggaagggagacaggggcacagagagacagaggttcatggactgaggcaaaggctgggccaggctcagcaacccaggcctcccgcaggcaggcagaggctgccctgtaacccatggagaccagaccaacagctctgatgagctccacagtggctgcagctgcgcctgcagctggggctgcctccaggaaggagtctccaggcagatggggcctgggggaggatcccacaggcgtgagcccctcgctccagtgccgcgtgtgcggagacagcagcagcgggaagcactatggcatctatgcctgcaacggctgcacgggcttcttcaagaggagcgtacggcggaggctcatctacaggtgccaggtgggggcagggatgtgccccgtggacaaggcccaccgcaaccagtgccaggcctgccggctgaagaagtgcctgcaggcggggatgaaccaggacgccgtgcagaaggagcgccagccgcgaagcacagcccaggtccacctggacagcatggagtccaacactgagtcccggccggagtccctggtggctcccccggccccggcagggcgcagcccacggggccccacacccatgtctgcagccagagccctgggccaccacttcatggccagccttataacagctgaaacctgtgctaagctggagccagaggatgctgatgagaatattgatgtcaccagcaatgaccctgagttcccctcctctccatactcctcttcctccccctgcggcctggacagcatccatgagacctcggctcgcctactcttcatggccgtcaagtgggccaagaacctgcctgtgttctccagcctgcccttccgggatcaggtacctaccggcctgcctgctggggagctaggctgggctggggtcaggcggcccactcgagtcaaccagacagggcacacacatccccacgccagtatgaatgcacacagcttggatggtgatggctggggacacacatacctctgattcagcgatggctggggtgcatctcagggatggtgacggtgggggtgcatgcatctctggcacagggatgatggtcggggtgcacacctaggagatgatgatggctagggacctacagggcccagggtcttcttaagttctggaagaccctcaggccctgcagacattctgtgggtaacaagtgacctgcacaccctgaacaggctgagtggctgactctaggcccccttggagcacaagtgcctacgacttcagggcttgcattttagttcaatctctccagctctgggccatccctctcggcttctaatgggcaagcagatctttcaggaaaaccaggaggagaggcatgaggagggtttgaggccctcagccagtctgtgtgctggggtggagcaactcagaagagtcaggccacaccacttgaatacactcaacttaggacactcatgaggcatgtctctgaggctgcccaacttccaatggctctgggcgttcctaaatgtcccagctgcagctctggatggaacccagtgtctcagatgataggcagctgagccggatggtgccaaatcccagagctctgagcctctggctgatgtcaggagagcattctcgggtcccaggacagcacttccattccttgggtgcctgagatggtggcagaggctccagactgagccagagaagctgtgtgtctgccataacaggcacccctgtctgagcacaggtgatcctgctggaagaggcgtggagtgaactctttctcctcggggccatccagtggtctctgcctctggacagctgtcctctgctggcaccgcccgaggcctctgctgccggtggtgcccagggccggctcacgctggccagcatggagacgcgtgtcctgcaggaaactatctctcggttccgggcattggcggtggaccccacggagtttgcctgcatgaaggccttggtcctcttcaagccagagacgcggggcctgaaggatcctgagcacgtagaggccttgcaggaccagtcccaagtgatgctgagccagcacagcattctagaattaagcggccgctgaattctaggttaagatggcccttgacattgagcaggtctt

SEQ ID NO: 6 - protein, 297 aa, Homo sapiens
METRPTALMSSTVAAAAPAAGAASRKESPGRWGLGEDPTGVSPSLQCRVCGDSSSGKHYGIYACNGCSGFFKRSVRRRLIYRCQVGAGMCPVDKAHRNQCQACRLKKCLQAGMNQDAVQNERQPRSTAQVHLDSMESNTESRPESLVAPPAPAGRSPRGPTPMSAARALGHHFMASLITAETCAKLEPEDADENIDVTSNDPEFPSSPYSSSSPCGLDSIHETSARLLFMAVKWAKNLPVFSSLPFRDQVPTGLPAGELGWAGVRRPTRVNQTGHTHPHASMNAHSLDGDGWGHTYL

SEQ ID NO: 7 - DNA, 215 bp, Homo sapiens
  misc_feature (1)...(215): n = A,T,C or G
actgncaggaactcctgccaccgccaccggctcccatggcccacatacccagtgggggtgccccagcagcgggggcagcccccatgggcccccagtattgcgtgtgcaaggtggagctgtcagtgagtggccagaacctactggaccgggatgttacctccaagtccgaccccttctgtgtcctctttacagagaacaatggcagatggatcgag

SEQ ID NO: 8 - DNA, 3686 bp, Homo sapiens
gaattcccacaaaggagtccagggtctcgctctgtcacacaggctggagtgcagtggtgtgatcttggctcatcgtaacctccacctcccgggttcaactgattctcatgcctcagcctcccgagtagctgggattacaggtggtgacttccaagagtgactccgtcggaggaaaatgactccccagtcgctgctgcagacgacactgttcctgctgagtctgctcttcctggtccaagccagcggaaccagacacacaggagcagcctccactacaaacccacaccagacctgcgcatctccatcgagaactccgaagaggccctcacagtccatgcccctttccctgcagcccaccctgcttcccgatccttccctgaccccaggggcctctaccacttctgcctctactggaaccgacatgctgggagattacatcttctctatggcaagcgtgacttcttgctgagtgacaaagcctctagcctcctctgcttccagcaccaggaggagagcctggctcagggccccccgctgttagccacttctgtcacctcctggtggagccctcagaacatcagcctgcccagtgccgccagcttcaccttctccttccacagtcctccccacacggccgctcacaatgcctcggtggacatgtgcgagctcaaaagggacctccagctgctcagccagttcctgaagcatccccagaaggcctcaaggaggccctcggctgcccccgccagccagcagttgcagagcctggagtcgaaactgacctctgtgagattcatgggggacatggtgtccttcgaggaggaccggatcaacgccacggtgtggaagctccagcccacagccggcctccaggacctgcacatccactcccggcaggaggaggagcagagcgagatcatggagtactcggtgctgctgcctcgaacactcttccagaggacgaaaggccggaggggggaggctgagaagagactcctcctggtggacttcagcagccaagccctgttccaggacaagaattccagccacgtcctgggtgagaaggtcttggggattgtggtacagaacaccaaagtagccaacctcacggagcccgtggtgctcaccttccagcaccagctacagccgaagaatgtgactctgcaatgtgtgttctgggttgaagaccccacattgagcagcccggggcattggagcagtgctgggtgtgagaccgtcaggagagaaacccaaacatcctgcttctgcaaccacttgacctactttgcagtgctgatggtctcctcggtggaggtggacgccgtgcacaagcactacctgagcctcctctcctacgtgggctgtgtcgtctctgccctggcctgccttgtcaccattgccgcctacctctgctccaggaggaaacctcgggactacaccatcaaggtgcacatgaacctgctgctggccgtcttcctgctggacacgagcttcctgctcagcgagccggtggccctgacaggctctgaggctggctgccgagccagtgccatcttcctgcacttctccctgctcacctgcctttcctggatgggcctcgaggggtacaacctctaccgactcgtggtggaggtctttggcacctatgtccctggctacctactcaagctgagcgccatgggctggggcttccccatctttctggtgacgctggtggccctggtggatgtggacaactatggccccatcatcttggctgtgcataggactccagagggcgtcatctacccttccatgtgctggatccgggactccctggtcagctacatcaccaacctgggcctcttcagcctggtgtttctgttcaacatggccatgctagccaccatggtggtgcagatcctgcggctgcgcccccacacccaaaagtggtcacatgtgctgacactgctgggcctcagcctggtccttggcctgccctgggccttgatcttcttctcctttgcttctggcaccttccagcttgtcgtcctctaccttttcagcatcatcacctccttccaaggcttcctcatcttcatctggtactggtccatgcggctgcaggcccggggtggcccctcccctctgaagagcaactcagacagcgccaggctccccatcagctcgggcagcacctcgtccagccgcatctaggcctccagcccacctgcccatgtgatgaagcagagatgcggcctcgtcgcacactgcctgtggcccccgagccaggcccagccccaggccagtcagccgcagactttggaaagcccaacgaccatggagagatgggccgttgccatggtggacggactcccgggctgggcttttgaattggccttggggactactcggctctcactcagctcccacgggactcagaagtgcgccgccatgctgcctagggtactgtccccacatctgtcccaacccagctggaggcctggtctctccttacaacccctgggcccagccctcattgctgggggccaggccttggatcttgagggtctggcacatccttaatcctgtgcccctgcctgggacagaaatgtggctccagttgctctgtctctcgtggtcaccctgagggcactctgcatcctctgtcattttaacctcaggtggcacccagggcgaatggggcccagggcagaccttcagggccagagccctggcggaggagaggccctttgccaggagcacagcagcagctcgcctacctctgagcccaggccccctccctccctcagccccccagtcctccctccatcttccctggggttctcctcctctcccagggcctccttgctccttcgttcacagctgggggtccccgattccaatgctgttttttggggagtggtttccaggagctgcctggtgtctgctgtaaatgtttgtctactgcacaagcctcggcctgcccctgagccaggctcggtaccgatgcgtgggctgggctaggtccctctgtccatctgggcctttgtatgagctgcattgcccttgctcaccctgaccaagcacacgcctcagaggggccctcagcctctcctgaagccctcttgtggcaagaactgtggaccatgccagtcccgtctggtttccatcccaccactccaaggactgagactgacctcctctggtgacactggcctagagcctgacactctcctaagaggttctctccaagcccccaaatagctccaggcgccctcggccgcccatcatggttaattctgtccaacaaacacacacgggtagattgctggcctgttgtaggtggtagggacacagatgaccgacctggtcactcctcctgccaacattcagtctggtatgtgaggcgtgcgtgaagcaagaactcctggagctacagggacagggagccatcattcctrcctgggaatcctggaagacttcctgcaggagtcagcgttcaatcttgaccttgaagatgggaaggatgttctttttacgtaccaattcttttgtcttttgatattaaaaagaagtacatgttcattgtagagaatttggaaactgtagaagagaatcaagaagaaaaataaaaatcagctgttgtaatcgcctarcaaaaaaaaaaaaaaaaaaaaaaaaaaaaaaaaaataaaaaaaaaaaa

SEQ ID NO: 9 - protein, 512 aa, Homo sapiens
MCELKRDLQLLSQFLKHPQKASRRPSAAPASQQLQSLESKLTSVRFMGDMVSFEEDRINATVWKLQPTAGLQDLHIHSRQEEEQSEIMEYSVLLPRTLFQRTKGRRGEAEKRLLLVDFSSQALFQDKNSSHVLGEKVLGIVVQNTKVANLTEPVVLTFQHQLQPKNVTLQCVFWVEDPTLSSPGHWSSAGCETVRRETQTSCFCNHLTYFAVLMVSSVEVDAVHKHYLSLLSYVGCVVSALACLVTIAAYLCSRRKPRDYTIKVHMNLLLAVFLLDTSFLLSEPVALTGSEAGCRASAIFLHFSLLTCLSWMGLEGYNLYRLVVEVFGTYVPGYLLKLSAMGWGFPIFLVTLVALVDVDNYGPIILAVHRTPEGVIYPSMCWIRDSLVSYITNLGLFSLVFLFNMAMLATMVVQILRLRPHTQKWSHVLTLLGLSLVLGLPWALIFFSFASGTFQLVVLYLFSIITSFQGFLIFIWYWSMRLQARGGPSPLKSNSDSARLPISSGSTSSSRI

SEQ ID NO: 10 - protein, 10 aa, Homo sapiens
EQKLISEEDL

SEQ ID NO: 11 - protein, 5 aa, Homo sapiens
LVPRG

SEQ ID NO: 12 - protein, 10 aa, Homo sapiens
SAWRHPQFGG

(19) United States
(12) Patent Application Publication
(10) Pub. No.: US 2003/0152963 A1
(43) Pub. Date: Aug. 14, 2003

(54) Human chromosome 15 and 16 bardet-biedl syndrome polynucleotides and polypeptides and methods of use

(76) Inventors: David Duhl, Oakland, CA (US); Susan W. Gorman, Santa Monica, CA (US)

Correspondence Address: DAVIS WRIGHT TREMAINE, LLP, 2600 CENTURY SQUARE, 1501 FOURTH AVENUE, SEATTLE, WA, 98101-1688, US

(21) Appl. No.: 10/263,230

(22) Filed: Oct. 2, 2002

Related U.S. Application Data
(63) Continuation of application No. 09/602,597, filed on Jun. 22, 2000, now abandoned.
(60) Provisional application No. 60/141,753, filed on Jun. 30, 1999.

(51) Int. Cl. C12Q 1/68; G01N 33/53; G01N 33/567; C07H 21/04; C12P 21/02; C12N 5/06; A61K 48/00; C12N 9/00

(52) U.S. Cl. 435/6; 435/7.2; 435/69.1; 435/320.1; 435/183; 435/325; 514/44; 530/388.26; 536/23.2

(57) ABSTRACT

Amino acid and nucleic acid sequences of Bardet-Biedl Syndrome Region (BBSR) that map to human chromosome 15 or 16 are disclosed. Compositions and methods related to the genes and proteins are useful in the study, diagnosis and treatment of a variety of diseases including BBS and related conditions including obesity, retinal degeneration, and disorders affecting the nervous system, the heart, the kidneys, and the like. Compositions include those comprising BBSR polypeptides and derivatives thereof, nucleotide sequences, expression cassettes, vectors, transformed cells and antibodies. Methods include those for expression and detection of BBSR nucleotides and polypeptides.

### TECHNICAL FIELD

This invention relates to the identification and recombinant expression of human chromosome 15 and 16 Bardet-Biedl Syndrome Region (BBSR) proteins.

### BACKGROUND OF THE INVENTION

Bardet-Biedl Syndrome (BBS) is a clinically and genetically heterogeneous autosomal recessive disorder characterized by obesity, polydactyly, hypogenitalism, retinal degeneration, mental retardation and heart and kidney abnormalities.

Elbedour et al. (1994) Am. J. Med. Genet. 52(2):164-169 have reported hypertrophy of the interventricular septum and dilated cardiomyopathy, in addition to other previously reported congenital heart defects associated with BBS.

BBS has been mapped to loci on several human chromosomes including chromosomes 3, 11, 15 and 16. These loci include the 3p12, 11q13, 15q22 and the 16q21 chromosomal sites; also referred to as the BBS3, BBS1, BBS4 and BBS2 loci, respectively. (Bruford et al. (1997) Genomics 41(1):93-9; Leppert et al. (1994) Nature Genet. 7:108-112; Carmi et al. (1995) Hum. Mol. Genet. 4:9-13; Kwitek-Black et al. (1993) Nature Genet. 5:392-396; Sheffield et al. (1994) Hum. Mol. Genet. 3:1331-1335; Beales et al. (1997) J. Med. Genet. 34(2):92-8.).

Attempts to associate particular phenotypes with particular BBS loci have been reported. For example, Beales et al. (1997) J. Med. Genet. 34(2) 92-8, reported that affected subjects linked to the BBS2 and 4 loci were significantly shorter than their parents, while those linked to the BBS1 locus were taller, indicating possible role for various BBS genes in influencing growth characteristics such as height. Carmi et al. (1995) Hum. Mol. Genet. 4.9-13, reported that BBS3 is associated with polydactyly of all four limbs while BBS4 polydactyly is mostly confined to the hands and that BBS4 is associated with early-onset morbid obesity, while BBS2 appears as the leanest form of BBS.

Reports of specific genes involving BBS loci are sparse. Zhu et al. (1998) Hum. Genet. 193(6):674-680 have reported identification of a human p70s6 kinase with a possible role in BBS1 which is mapped to 11q13. Hoang et al. (1998) Genomics:52 (2): 219-222, have reported cloning of a C-terminal kinesin (KIFC3) that maps to human 1⁶q 13-q21 within the BBS2 region.

BBS and associated disorders have many serious effects on humans. There is a need for identifying compositions that are useful in diagnosis and treatment of such disorders.

### SUMMARY OF THE INVENTION

The present invention discloses amino acid and nucleic acid sequences of human chromosome 15 and 16 Bardet-Biedl Syndrome Region (BBSR). The corresponding genes are referred to as Gene X, plasmolipin-like protein (PLP), ORPH-PPAR (PPAR), NT2 neuronal precursor-like (NTPL), and a seven transmembrane domain protein. The new genes and proteins are useful in the study, diagnosis and treatment of a variety of diseases including BBS and related conditions. Other indications that can be treated by the BBSR nucleotides, proteins and/or BBSR polypeptides, or agonists or antagonists include obesity, retinal degeneration, and disorders affecting the central nervous system, the heart, the kidneys, and the like.

Compositions and methods for expressing and using BBSR nucleotides and proteins are provided. The compositions comprise BBSR polypeptides and derivatives thereof, nucleotide sequences, expression cassettes, vectors, transformed cells and antibodies. Methods for the expression and detection of BBSR nucleotides and polypeptides and compositions for the treatment of BBS related conditions are provided.

The invention further provides (a) a polynucleotide encoding amino acids from about 1 or about 2 to about 254 of SEQ ID NO:2; (b) a polynucleotide encoding amino acids from about 1 or about 2 to about 218 of SEQ ID NO:4; (c) a polynucleotide encoding amino acids from about 1 or about 2 to about 297 of SEQ ID NO:6; (d) a polynucleotide encoding amino acids from about 1 or about 2 to about 513 of SEQ ID NO:9; (e) a polynucleotide comprising SEQ ID NO:7; (f) the polynucleotide complement of the polynucleotide of any one of (a) through (e); and (g) a polynucleotide at least 90% identical to the polynucleotide of any one of (a) through (e).

The invention still further provides a method of making a recombinant vector comprising inserting a nucleic acid molecule of (a) through (g) into a vector in operable linkage to a promoter; a recombinant vector produced thereby; a method of making a recombinant host cell comprising introducing the recombinant vector into a host cell; a recombinant host cell produced by this method; and a recombinant method of producing a polypeptide, comprising culturing the recombinant host cell under conditions such that the polypeptide is expressed, and recovering the polypeptide

The invention also provides an isolated polypeptide comprising an amino acid sequence from (a) about 1 or about 2 to about 254 of SEQ ID NO:2; (b) about 1 or about 2 to about 218 of SEQ ID NO:4; (c) about 1 or about 2 to about 297 of SEQ ID NO:6; or (d) about 1 or about 2 to about 513 of SEQ ID NO:9; an isolated polypeptide wherein, except for at least one conservative amino acid substitution, the polypeptide has an amino acid sequence of one of (a) through (d); and an isolated polypeptide comprising amino acids at least 95% identical to an amino acid sequence of one of (a) through (d).

### BRIEF DESCRIPTION OF THE DRAWINGS

FIG. 1 provides the nucleotide and amino acid sequence of the human Gene X protein (SEQ ID NO:1,2).

FIG. 2 provides the nucleotide and amino acid sequence of the human PLP cDNA (SEQ ID NO:3, 4)

FIG. 3 provides the nucleotide and amino acid sequence of the human PPAR cDNA (SEQ ID NO:5, 6).

FIG. 4 provides the nucleotide sequence of the human NTPL cDNA (SEQ ID NO:7).

FIG. 5 provides the nucleotide and amino acid sequence of a human seven transmembrane receptor protein (SEQ ID NO:8, 9).

### DETAILED DESCRIPTION OF THE INVENTION

Compositions and methods for expressing and using BBSR nucleotides, proteins and polypeptides are provided. The compositions and methods find use in the treatment of BBS and BBS-related conditions including obesity, retinal degeneration, mental retardation, central nervous system disorders, heart and kidney abnormalities, and the like.

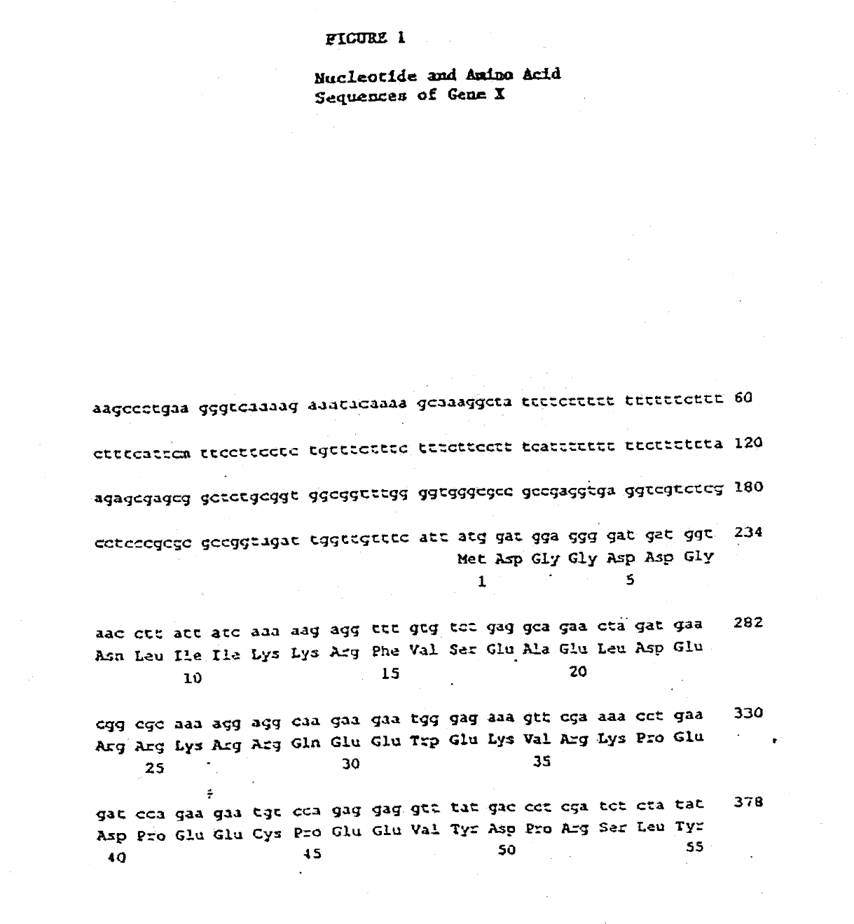

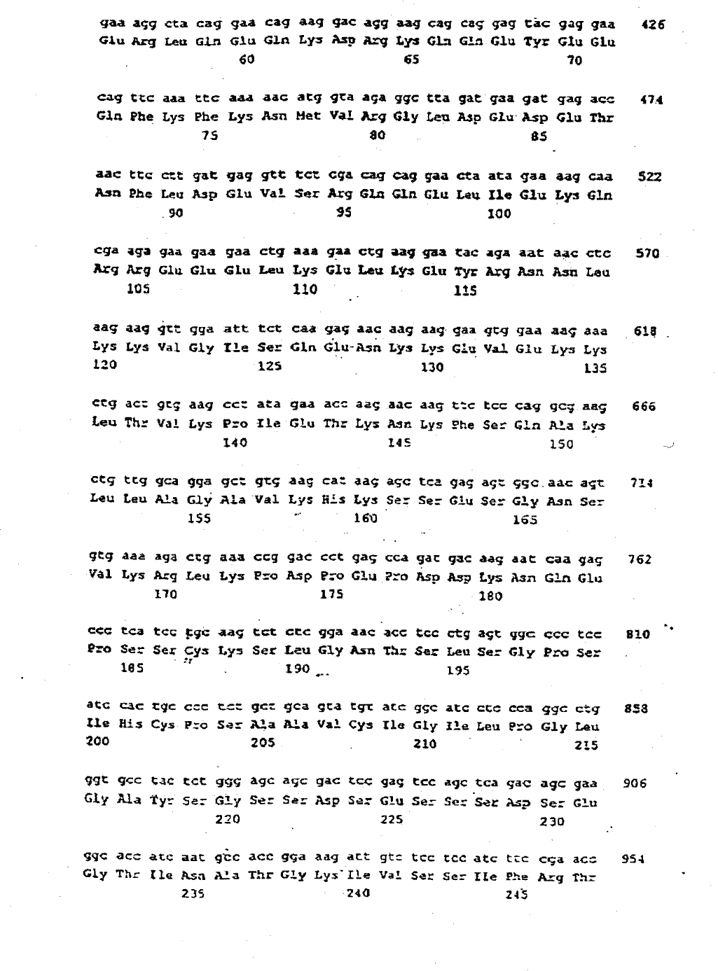

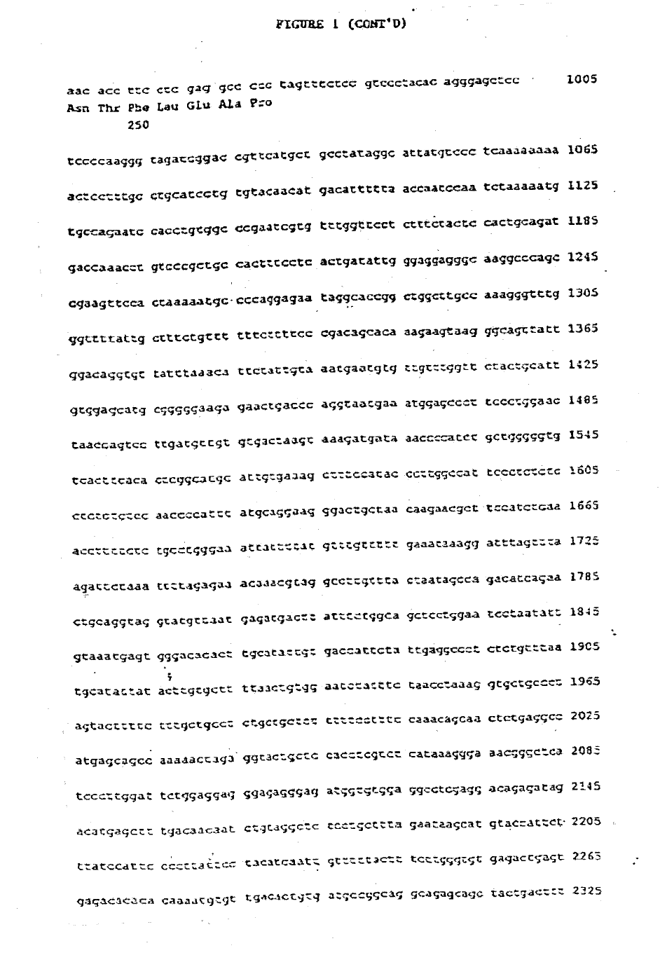

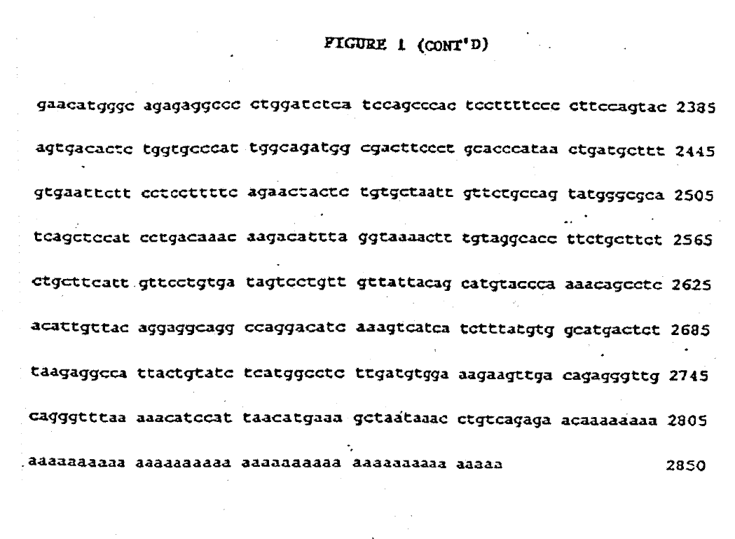

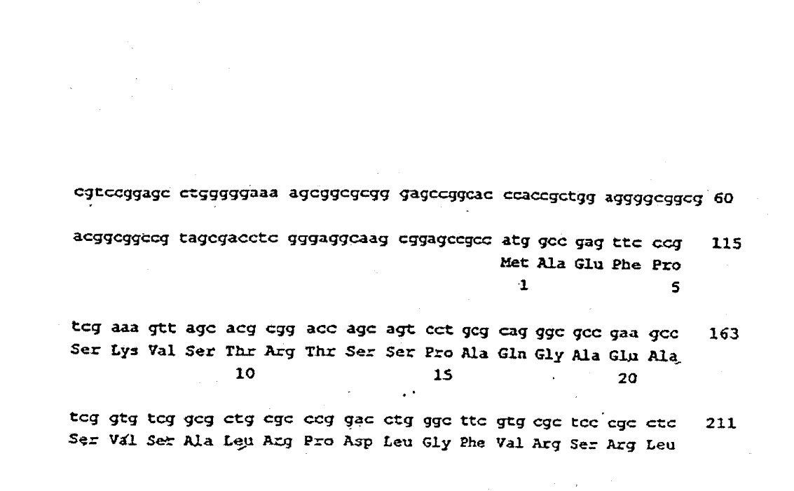

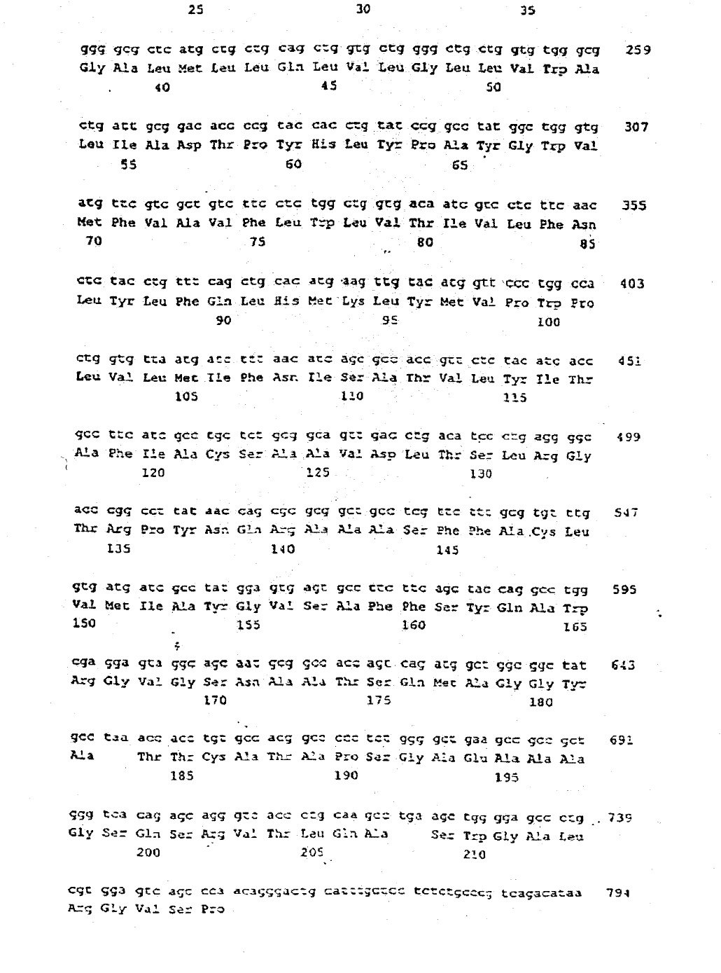

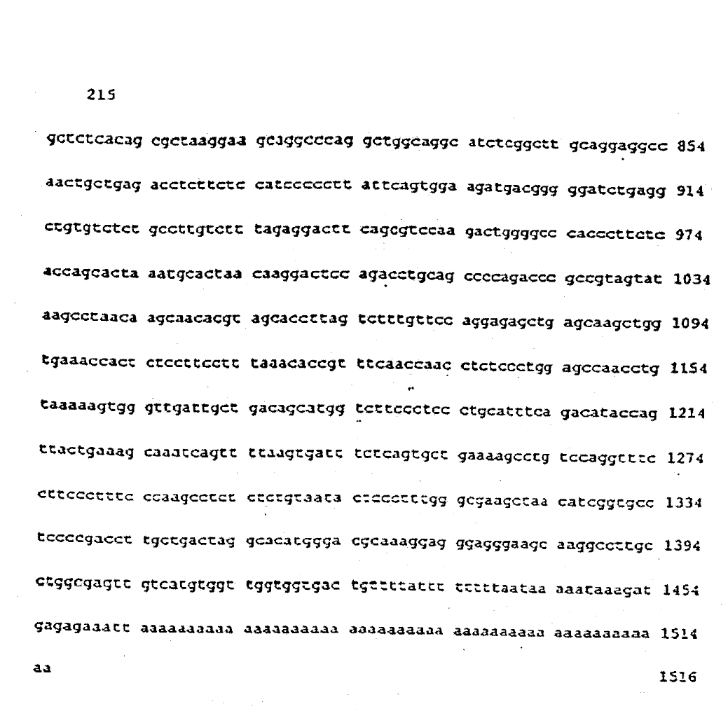

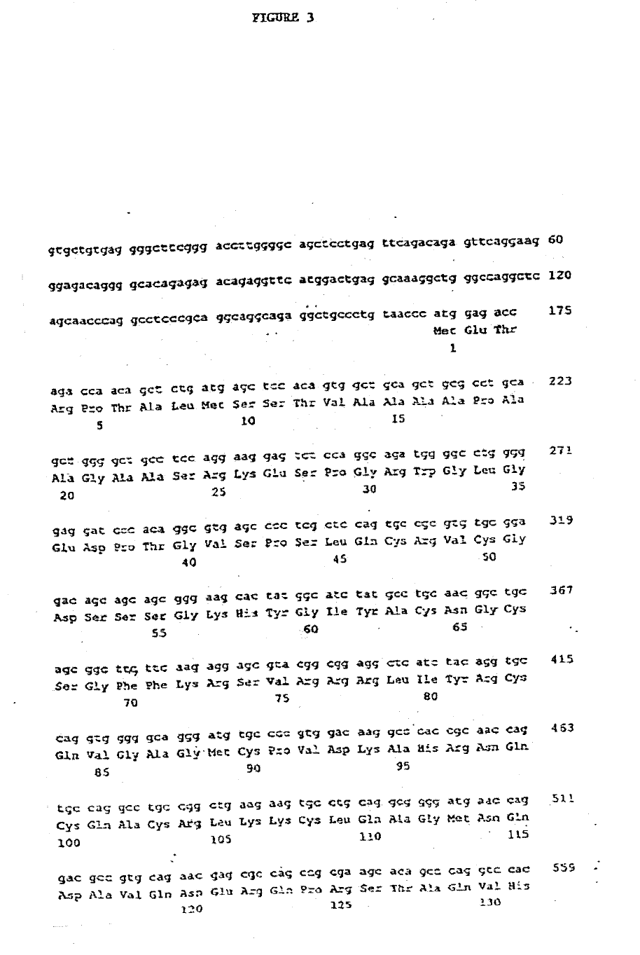

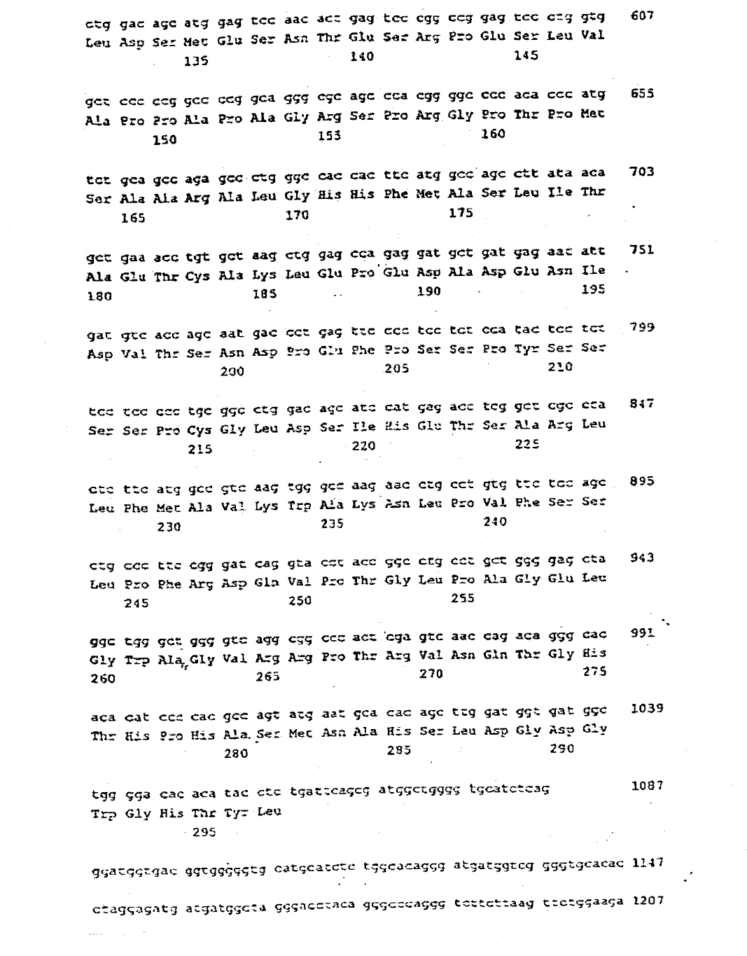

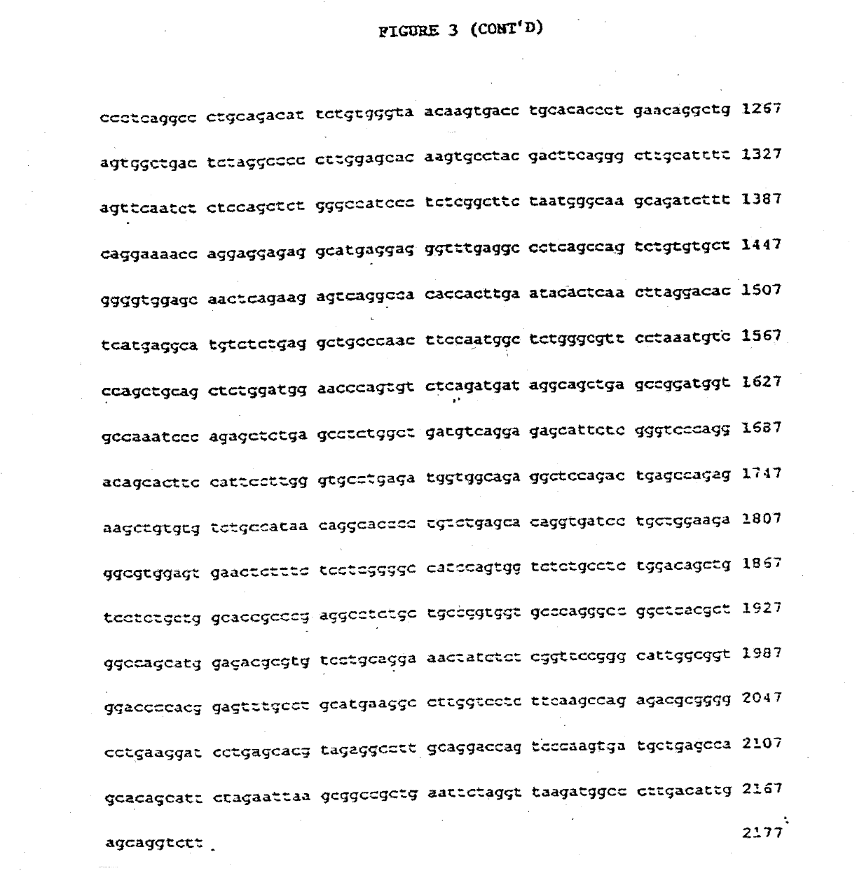

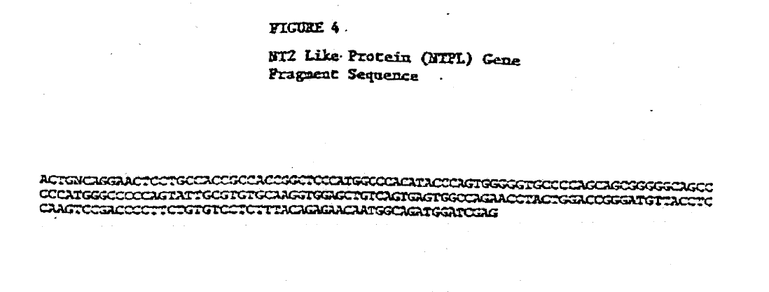

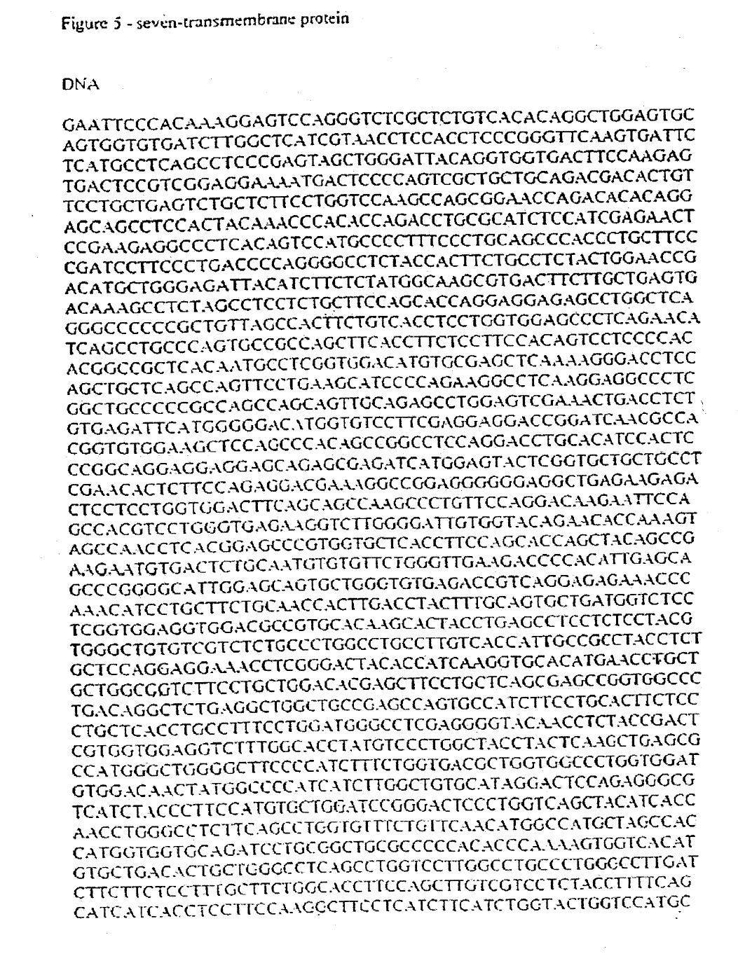

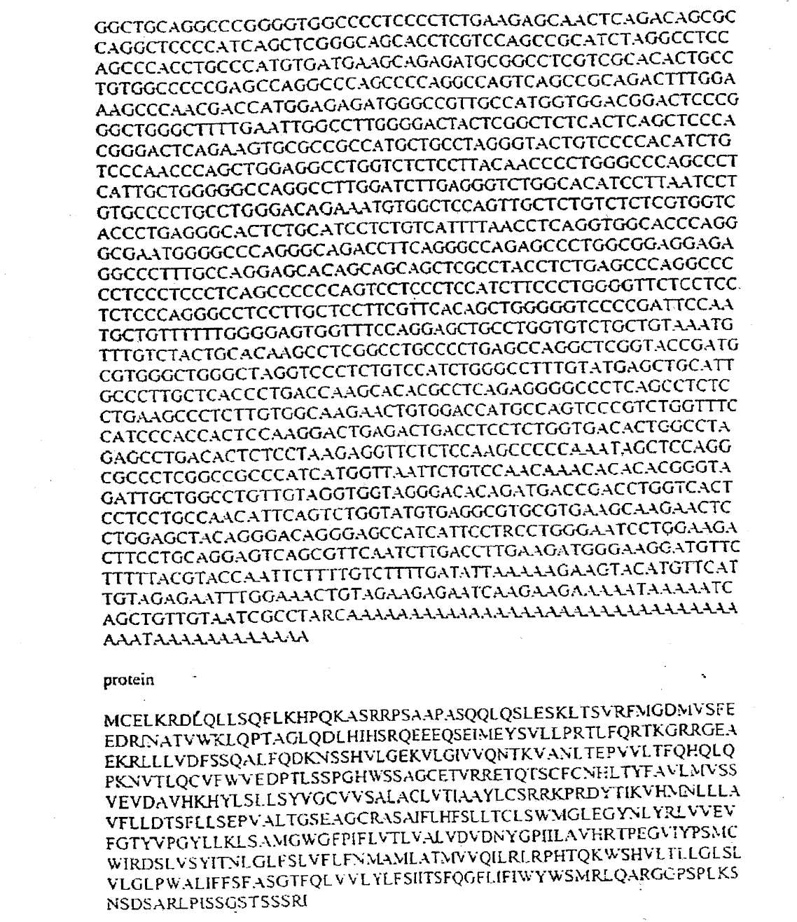

More particularly, new genes, and polypeptides encoded by the genes have been identified that are useful in the treatment of these and a variety of other conditions. The human BBSR polypeptides and cDNAs are provided in FIGS. 1-5 (SEQ ID NO: 1-9).

The compositions and methods of the invention can be used for the treatment and diagnosis of BBS, a disorder having a clinical manifestation of BBS, or any disorder that shares a clinical manifestation of BBS, so long such disorders can be diagnosed and/or treated by the methods and compositions of the invention, in a clinically or experimentally determinable manner.

The invention provides isolated nucleic acid molecules comprising a nucleotide sequence encoding the BBSR polypeptides whose amino acid sequences are provided in FIGS. 1-5, or a variant or fragment of the polypeptides. Furthermore, polynucleotides comprising antisense sequences for BBSR proteins are provided.

The BBSR sequences provided in FIGS. 1-5 (SEQ ID NO:1-9) correspond to Gene X, plasmolipin-like protein (PLP), ORPH-PPAR (PPAR), NT2 neuronal precursor-like protein (NTPL), and 7-transmembrane receptor protein, respectively.

For Gene X, the invention provides a 2850 bp cDNA (SEQ ID NO:1), which was isolated from human brain frontal cortex, and encodes an open reading frame of 762 bp (encompassing bases 214-976). Northern analysis showed that the transcript of Gene X is expressed in brain (cerebellum, cerebral cortex, medulla, spinal cord, occipital lobe, frontal lobe, temporal lobe and putamen), heart, skeletal muscle, spleen, testis, pancreas. Very low expression was detected in thymus, ovary, small intestine, colon, peripheral blood leukocytes, placenta, lung, liver and kidney. The gene is located on chromosome 16.

For plasmolipin-like protein, the invention provides a 1516 bp cDNA (SEQ ID NO:3) from human brain that encodes an open reading frame of 545 bp (encompassing bases 210-755). At the nucleotide level, this sequence contains about 85% identity with human NTII-11 nerve protein and rat plasmolipin. At the amino acid level the sequence has about 89% identity with rat plasmolipin and with NTII-11. The transcript of this novel gene is expressed in brain, kidney, lung, pancreas, spleen, prostate, heart, testis, small intestine, liver, colon, skeletal muscle, placenta and ovary, but is not visible on Northern blots of thymus and peripheral blood leukocytes.

Plasmolipin is a proteolipid found on the apical surface of tubular epithelial cells of the kidney and in myelinated tracts of the brain. Addition of plasmolipin to lipid bilayers induces the formation of ion channels, which are voltage-dependent and K(+)-selective. The PLP nucleic acids and proteins of the invention find use in membrane trafficking, gap junction formation, ion transport and cell volume regulation (U.S. Pat. No. 5,843,714). In addition, plasmolipin transcript levels correlate with myelination of nerve cells, as crushed nerves undergoing regeneration displayed increased transcript levels.

Proteins involved in ion transport play vital roles in excitable cells and tissues, including muscle and nerve, in which ionic changes are primarily associated with electrophysiological responses such as generation of action potentials and/or excitation-contraction coupling. It is also known that proteins involved in ion transport play important roles in tissues in which ion transport is a primary physiological function, such as kidney.

The novel plasmolipin-like gene of this invention (PLP) is implicated in Bardet-Biedl syndrome (BBS) since it falls within the region containing the chromosome 16 BBS gene. The expression pattern of the PLP gene of the invention is consistent with the pleiotropic manifestations of BBS. Furthermore, since plasmolipin is involved in ion transport and myelination, the PLP sequences of the invention may be involved in the health and proper maintenance of nerve cells/fibers, and are particularly useful for diagnosis and treatment of disorders associated with the nervous system, including central and peripheral nervous systems. Examples of such disorders include but are not limited to BBS, Laurence-Moon-Bardet-Biedl syndrome, leukodystrophy, multiple sclerosis, Charcot-Marie-Tooth neuropathy type 1a, pressure neuropathy HNPP and Dejarine-Sottas disease.

For the new transcription factor, the invention provides a 2177 bp cDNA (SEQ ID NO:5) from human retina that encodes an open reading frame of 891 bp (encompassing bases 167-1057). The transcript of the transcription factor gene of the invention appeared ˜1.5 kb on Northern and was expressed in brain (cerebellum, cerebral cortex, medulla, spinal cord, occipital lobe, frontal lobe, temporal lobe and putamen), heart, placenta, lung, liver, skeletal muscle, kidney, pancreas, spleen, thymus, prostate, testis, ovary, small intestine, colon, and peripheral blood leukocytes. The transcript contains an open reading frame of 891 bp encompassing bases 167-1057 and predicts a protein of 297 amino acids.

The sequence exhibited some homology to an apolipoprotein A1 regulatory protein. Apo A1 expression is significant in cholesterol metabolism and atheroscleorosis, particularly in light of the role of apo A1 in the reverse transport of cholesterol from peripheral tissues such as coronary arteries to the liver, the primary site of cholesterol metabolism for excretion. The sequences of the invention are particularly useful for the diagnosis and treatment of disorders associated with cholesterol homeostasis, for example, but not limited to obesity, atheroscleorosis, and the like. Furthermore, the expression pattern of the gene of the invention is consistent with the pleiotropic manifestations of BBS involving obesity and cardiac disorders. It is recognized that the sequences of the invention may be used to modulate cholesterol metabolism.

For the NT2 neuronal precursor-like clone (SEQ ID NO:7), the invention provides an isolated 214 base pair fragment located on BAC 17354. Northern blots utilizing a 126 base pair probe corresponding to this fragment identified a transcript of about 2.3 kb that was present in brain (cerebellum, cerebral cortex, medulla, spinal cord, occipital lobe, frontal lobe, temporal lobe and putamen), heart, placenta, lung, liver skeletal muscle, kidney, pancreas, spleen, prostate, ovary, small intestine, colon, and peripheral blood leukocytes. A slightly larger transcript, about 2.4 kb, was present in testis and no expression appeared in thymus RNA.

For human chromosome 16 seven transmembrane receptor protein, the invention provides a 3686 bp cDNA fragment (SEQ ID NO:8) from human brain (frontal cortex) that encodes an open reading frame of 1539 bp (encompassing bases 658-2193). This gene is located on human chromosome 16. Hydrophobicity analysis indicates 7 membrane spanning regions. On Northern blots, this novel gene shows a transcript of ˜4.4 kb that is expressed in brain (cerebellum, cerebral cortex, medulla, spinal cord, occipital lobe, frontal lobe, temporal lobe and putamen), heart, placenta, lung, liver, skeletal muscle, kidney, pancreas, spleen, thymus, prostate, testis, ovary, small intestine, colon and peripheral blood leukocytes. A less abundant transcript of ˜6.2 is also present in all the tissues listed above. However, the ratio of expression of the two transcripts varied from tissue to tissue. For example, in heart the 4.4 kb transcript appeared to be expressed 10× more than the ˜6.2 kb transcript, but in brain this ratio was closer to 50×. The transcript encodes a minimum of 10 exons.

This gene may relate to Bardet-Biedl syndrome since it falls within the region containing the chromosome 16 BBS gene. The 7-transmembrane domain gene can be used to develop drug treatments and therapies for obesity and retinal degeneration

Polypeptides of the invention encompass the sequences set forth herein as well as derivatives, analogs and variants thereof. Unless otherwise indicated, variants include substantially homologous proteins having at least about 60-65%, typically at least about 70-75%, more typically at least about 80-85%, and most typically at least about 90, 91, 92, 93, 94, 95, 96, 97, 98 or 99% or more homology to one of SEQ ID NO:2, 4, 6 or 9. It is recognized that amino acid substitutions may be made, particularly conservative substitutions. See, Bowie et al. (1990) Science 247:1306-1310. A variant polypeptide can differ in amino acid sequence by one or more substitutions, deletions, insertions, inversions, fusions and truncations or a combination of any of these. Variants can be naturally-occurring or can be made by recombinant means or chemical synthesis. Variant polypeptides may be fully functional or lack function in one or more activities.

Amino acids in the protein that are essential for function can be identified by site-directed mutagenesis, alanine-scanning mutagenesis (Cunningham et al. (1989) Science 244:1081-1085), etc. The resulting mutant molecules are then tested for biological activity. Critical sites for receptor binding can be determined. See, for example, Smith et al. (1992) J. Mol. Biol. 224:899-904; de Vos et al. (1992) Science 255:306-312.

Another aspect of the invention is a chimeric polypeptide comprising a BBSR polypeptide, or fragment thereof and a polypeptide of interest. Similarly, the invention provides a chimeric polypeptide comprising a BBSR polypeptide, or fragment thereof, fused to a polypeptide of interest. Nucleotide sequences encoding chimeric BBSR proteins and polypeptides are also provided.

Yet another object of the invention is to provide polynucleotides that encode the mutants, fragments, and derivatives, as well as the native BBSR proteins and polypeptides. These polynucleotides can be operably linked to heterologous promoters to form expression cassettes. The expression cassettes can be introduced into suitable host cells for expression of BBSR proteins and/or polypeptides and derivatives thereof.

The invention encompasses polynucleotide sequences having at least 65% sequence identity to SEQ ID NO:1, 3, 5, 7, or 8 as determined using algorithms known to those of ordinary skill in the art. A preferred but non-limiting example of a suitable algorithm is the Smith-Waterman homology search algorithm as implemented in MSPRCH program (Oxford Molecular) using an affine gap search with the following search parameters: gap open penalty of 12, and gap extension penalty of 1. For the purpose of encoding polypeptides that vary in respect to SEQ ID NO:2, 4, 6, or 9 as described herein, the polypeptide encoded by the polynucleotides having the percent homology described above is tested for retention of biological characteristics of the native protein, and significant variation from the sequence of SEQ ID NO:1, 3, 5, or 8 may be permitted as long as the protein retains such characteristics. However, if the polynucleotide variant is used as a probe for, for example, mRNA corresponding to SEQ ID NO:1, 3, 5, 7, or 8, then percent homology should be maximized to allow specific detection of the mRNA.

Another object of the invention is to provide a transformed cell transiently expressing or having stably incorporated into its genome an expression vector comprising a promoter operably linked to a nucleotide sequence encoding a BBSR protein or polypeptide, or a fragment, derivative, mutant or fusion thereof.

The invention further provides methods for treating BBSR protein modulated disorders, including but not limited to Bardet-Biedl Syndrome, retinal degeneration including retinitis pigmentosa, obesity, mental retardation, renal abnormalities, diabetes and cardiovascular abnormalities. The methods comprise administering a therapeutically effective amount of a BBSR protein or polypeptide, or a derivative thereof to a subject in need of such treatment. In still another aspect, the invention provides a composition comprising BBSR protein or polypeptide or an active derivative thereof, and a pharmaceutically acceptable carrier.

The compositions of the invention comprise amino acid and nucleotide sequences for BBSR proteins. Such compositions have several uses including diagnosis and treatment of other BBSR protein-modulated disorders.

“BBSR protein-modulated disorders” and “BBSR protein modulated-disorders” include BBS and its various clinical manifestations including but not limited to obesity, hypogenitalism, retinal degeneration, retinis pigmentosa, polydactyly, brachydactyly, syndactyly, mental retardation, cardiovascular and renal abnormalities, and the like.

BBSR protein-modulated disorders and BBSR protein modulated-disorders also include disorders other than BBS which include a clinical manifestation associated with BBS. For example, several human diseases exist which manifest an obesity phenotype, including but not limited to Ahistroem syndrome, polycystic ovarian disease, Usher's, Carpenter, Prader Willi, Cohen, and Morgagni-Stewart-Monel Syndromes.

Other Examples include, in addition to BBS, other human diseases characterized by retinal degeneration including, without limitation, Bassen-Kornzweig syndrome (abetalipoproteinemia), Best disease (vitelliform dystrophy), choroidemia, gyrate atrophy, congenital amaurosis, Refsum syndrome, Stargardt disease and Usher syndrome. Other retinopathies that may benefit from administration of the compositions of the invention include age-related macular degeneration (dry and wet forms), diabetic retinopathy, peripheral vitreoretinopathies, photic retinopathies, surgery-induced retinopathies, viral retinopathies (such as HIV retinopathy related to AIDS), ischemic retinopathies, retinal detachment and traumatic retinopathy retinal

The methods and compositions of the invention can be used for the treatment and diagnosis of BBS, a disorder having a clinical manifestation of BBS, or any disorder that shares a clinical manifestation of BBS, so long such disorders can be diagnosed and/or treated by the methods and compositions of the invention, in a clinically or experimentally determinable manner.

“Stringency” refers to conditions in a hybridization reaction that favor association of very similar sequences over sequences that differ. Factors affecting the stringency of hybridization are well known to those skilled in the art and are discussed in Sambrook et al., Molecular Cloning: A Laboratory Manual, Second Edition, Volume 2, Chapter 9, at page 9.50.

In general, convenient hybridization temperatures in the presence of 50% formamide are 42° C. for a probe with is 95% to 100% homologous to the target fragment, 37° C. for 90% to 95% homology, and 32° C. for 85% to 90% homology. For lower homologies, formamide content should be lowered and temperature adjusted accordingly, using the equation above. If the homology between the probe and the target fragment are not known, the simplest approach is to start with both hybridization and wash conditions, which are nonstringent. If nonspecific bands or high background are observed after autoradiography, the filter can be washed at high stringency and reexposed. If the time required for exposure makes this approach impractical, several hybridization and/or washing stringencies should be tested in parallel.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

A composition containing A is “substantially free of” B when at least about 85% by weight of the total A+B in the composition is A. Preferably, A comprises at least about 90% by weight of the total of A+B in the composition, more preferably at least about 95% or even 99% by weight.

Nucleic Acid BBSR Protein Probe Assays

mRNA levels in different cell types can be detected with nucleic acid probe assays For example, PCR, branched DNA probe assays, or blotting techniques utilizing nucleic acid probes substantially identical or complementary to SEQ ID NO:1, 3, 5, 7, or 8 can determine the presence of BBSR protein cDNA or mRNA.

For genomic analysis or detection of denatured DNA, the nucleic acid probes will hybridize to a nucleotide sequence encoding the amino acid sequence shown in SEQ ID NO:2, 4, 6, or 9, or the complement of a sequence encoding SEQ ID NO:1, 3, 5, 7, or 8. Though many different nucleotide sequences will encode the amino acid sequences, SEQ ID NO:2, 4, 6, or 9 is preferred because it is the actual sequence expressed in the human cells as disclosed herein. For single-stranded cDNA detection, the nucleic acid probe will hybridize to the complement of a nucleotide sequence encoding the amino acid sequence of SEQ ID NO:2, 4, 6, or 9 or to a complement of SEQ ID NO:1, 3, 5, 7, or 8. For mRNA detection, the nucleic acid probe will hybridize to SEQ ID NO:1, 3, 5, 7, or 8 or to a nucleotide sequence encoding the amino acid sequence of SEQ ID NO:2, 4, 6, or 9. The nucleic acid probe sequences need not be identical to SEQ ID NO:1, 3, 5, 7, or 8 or complements thereof.

Probes are typically at least about 15 to 20 nucleotides, more preferably at least about 30 nucleotides. The probes may be produced by synthetic procedures, such as the triester method of Matteucci et al. (1981) J. Am. Chem. Soc. 103:3185, or according to Urdea et al. (1983) Proc. Natl. Acad. Sci. USA 80:7461, or using commercially available automated oligonucleotide synthesizers. One example of a nucleotide hybridization assay is described in Urdea et al. PCT WO92/02526 and Urdea et al. U.S. Pat. No. 5,124,246, herein incorporated by reference. Other methods of hybridization and detection are known to those skilled in the art.

Alternatively, the Polymerase Chain Reaction (PCR) is another well-known means for detecting small amounts of target nucleic acids. The assay is described in Mullis et al. (1987) Meth. Enzymol. 155:335-350; U.S. Pat. No. 4,683,195; and U.S. Pat. No. 4,683,202, incorporated herein by reference. Also, mRNA, cDNA and genomic DNA can be detected by traditional blotting techniques described in Sambrook et al. (1989) Molecular Cloning: A Laboratory Manual (New York, Cold Spring Harbor Laboratory).

BBSR Proteins

By “BBSR proteins” is meant the proteins and polypeptides encoded by SEQ ID NO: 1, 3, 5, 7 and 8, preferably the polypeptides having the amino acid sequence of SEQ ID NO: 2, 4, 6 or 9.

Reference to the individual BBSR proteins disclosed herein is intended to be construed to include BBSR proteins of any origin which are substantially homologous to and which are biologically equivalent to the BBSR proteins characterized and described herein. Such substantially homologous proteins may be native to any tissue or species and, similarly, biological activity can be characterized in any of a number of biological assay systems.

The term “biologically equivalent” is intended to mean that the compositions of the present invention are capable of demonstrating some or all of the same biological properties in a similar fashion, not necessarily to the same degree as the BBSR as described herein or recombinantly produced human BBSR of the invention.

By “substantially homologous” it is meant that the degree of homology of human BBSR protein to BBSR protein from any species is greater than that between a BBSR of the invention and any previously described corresponding BBSR protein.

Sequence identity or percent identity is intended to mean the percentage of same residues between two sequences, referenced to human BBSR protein when determining percent identity with non-human BBSR protein, referenced to BBSR protein when determining percent identity with non-BBSR proteins, when the two sequences are aligned using the Clustal method (Higgins et al., Cabios 8.189-191 (1992)) of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 (Dayhoff et al., in Atlas of Protein Sequence and Structure, Dayhoff, Ed., NDRF, Washington, Vol. 5, suppl. 3, p. 3 4 5, 1978).

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to human BBSR protein when determining percent conservation with non-human BBSR protein, and referenced to BBSR when determining percent conservation with non-BBSR protein. Conservative amino acid changes satisfying this requirement are: R-K; E-D, Y-F, L-M; V-I, Q-H.

The invention provides BBSR proteins or variants thereof having one or more polymers covalently attached to one or more reactive amino acid side chains. By way of example, not limitation, such polymers include polyethylene glycol (PEG), which can be attached to one or more free cysteine sulfhydryl residues, thereby blocking the formation of disulfide bonds and aggregation when the protein is exposed to oxidizing conditions. In addition, pegylation of BBSR proteins and/or muteins is expected to provide such improved properties as increased half-life, solubility, and protease resistance. BBSR proteins and/or muteins may alternatively be modified by the covalent addition of polymers to free amino groups such as the lysine epsilon or the N-terminal amino group. It will be apparent to one skilled in the art that the methods for assaying BBSR protein biochemical and/or biological activity may be employed in order to determine if modification of a particular amino acid residue affects the activity of the protein as desired.

It may be advantageous to improve the stability of BBSR protein by modifying one or more protease cleavage sites. Thus, the present invention provides BBSR protein variants in which one or more protease cleavage site has been altered by, for example, substitution of one or more amino acids at the cleavage site in order to create as BBSR protein variant with improved stability. Such improved protein stability may be beneficial during protein production and/or therapeutic use.

Suitable protease cleavage sites for modification are well known in the art and likely will vary depending on the particular application contemplated. For example, typical substitutions would include replacement of lysines or arginines with other amino acids such as alanine. Preferred sites to substitute would include dibasic or tribasic sites within two residues of a proline. The loss of biological activity can be tested for the appropriate BBSR protein.

BBSR protein can also include hybrid and modified forms of BBSR protein including fusion proteins and BBSR fragments and hybrid and modified forms in which certain amino acids have been deleted or replaced and modifications such as where one or more amino acids have been changed to a modified amino acid or unusual amino acid and modifications such as glycosylations so long as the hybrid or modified form retains the biological activity of BBSR.

Fusion proteins comprising BBSR protein or a biologically active or antigenic fragment thereof can be produced using methods known in the art. Such fusion proteins can be used therapeutically or can be produced in order to simplify the isolation and purification procedures. Histidine residues can be incorporated to allow immobilized metal affinity chromatography purification. Residues EQKLISEEDL contain the antigenic determinant recognized by the myc monoclonal antibody and can be incorporated to allow myc monoclonal antibody-based affinity purification. A thrombin cleavage site can be incorporated to allow cleavage of the molecule at a chosen site; a preferred thrombin cleavage site is residues LVPRG. Purification of the molecule can be facilitated by incorporating a sequence, such as residues SAWRHPQFGG, which binds to paramagnetic streptavidin beads. Such embodiments are described in WO 97/25345, which is incorporated by reference.

The invention also includes fragments of BBSR proteins. Such fragments can be prepared from the protein by standard biochemical methods or by expressing a polynucleotide encoding the fragment. Also included with the scope of the invention are BBSR protein molecules that differ from native BBSR proteins by virtue of changes in biologically active sites.

Also included within the meaning of substantially homologous is any BBSR protein which may be isolated by virtue of cross-reactivity with antibodies to the BBSR described herein or whose encoding nucleotide sequences including genomic DNA, mRNA or cDNA may be isolated through hybridization with the complementary sequence of genomic or subgenomic nucleotide sequences or cDNA of the BBSR protein herein or fragments thereof. It will also be appreciated by one skilled in the art that degenerate DNA sequences can encode human BBSR proteins and these are also intended to be included within the present invention as are allelic variants of BBSR proteins.

The DNA encoding BBSR proteins can be engineered to take advantage of preferred codon usage of host cells. Codon usage in Pseudomonias aeruginosa is described in, for example, West et al., Nucleic Acids Res. 11.9323-9335 (1988). Codon usage in Saccharomyces cerevisiae is described in, for example, Lloyd et al., Nucleic Acids Res. 20:5289-5295 (1992). Codon preference in Corynebacteria and a comparison with E. coli preference is provided in Malubres et al., Gene 134:15-24 (1993). Codon usage in Drosophila melanogaster is described in, for example, Akashi, Genetics 136:927-935 (1994).

Any suitable expression vector may be employed to produce recombinant human BBSR proteins such as expression vectors for use in insect cells. Baculovirus expression systems can also be employed.

The present invention includes nucleic acid sequences including sequences that encode human BBSR proteins. Also included within the scope of this invention are sequences that are substantially the same as the nucleic acid sequences encoding BBSR proteins. Such substantially the same sequences may, for example, be substituted with codons more readily expressed in a given host cell such as E. coli according to well known and standard procedures. Such modified nucleic acid sequences are included within the scope of this invention.

Specific nucleic acid sequences can be modified by those skilled in the art and, thus, all nucleic acid sequences that code for the amino acid sequences of BBSR proteins can likewise be so modified. The present invention thus also includes nucleic acid sequence which will hybridize with all such nucleic acid sequences—or complements of the nucleic acid sequences where appropriate—and encode a polypeptide having the neuronal cell survival promoting activities disclosed herein. The present invention also includes nucleic acid sequences that encode polypeptides that have neuronal cell survival promoting activity and that are recognized by antibodies that bind to BBSR proteins.

The present invention also encompasses vectors comprising expression regulatory elements operably linked to any of the nucleic acid sequences included within the scope of the invention. This invention also includes host cells of any variety that have been transformed with vectors comprising expression regulatory elements operably linked to any of the nucleic acid sequences included within the scope of the present invention.

Expression of BBSR Protein and BBSR Polypeptides

Preferably, BBSR proteins and polypeptides are produced by recombinantly engineered host cells. These host cells are constructed by the introduction of a expression vector comprising a promoter operably linked to a BBSR protein or polypeptide coding sequence.

Such coding sequences can be constructed by synthesizing the entire gene or by altering existing BBSR protein or polypeptide coding sequences. BBSR polypeptides can be divided into four general categories: mutants, fragments, fusions, and the native BBSR polypeptides. The native BBSR polypeptides are those that occur in nature. The amino acid sequence of such polypeptides may vary slightly from SEQ ID NO:2, 4, 6, and 9. The native BBSR protein and BBSR polypeptide coding sequence can be selected based on the amino acid sequence shown in SEQ ID NO:2, 4, 6, and 9. For example, synthetic genes can be made using codons preferred by the host cell to encode the desired polypeptide. (See Urdea et al. (1983) Proc. Natl. Acad. Sci. USA 80:7461). Alternatively, the desired native BBSR polypeptide coding sequences can be cloned from nucleic acid libraries. Techniques for producing and probing nucleic acid sequence libraries are described, for example, in Sambrook et al. (1989) Molecular Cloning: A Laboratory Manual (New York, Cold Spring Harbor Laboratory). Other recombinant techniques, such as site specific mutagenesis, PCR, enzymatic digestion and ligation, can also be used to construct the desired BBSR protein or polypeptide coding sequence.

The native BBSR polypeptide coding sequences can be modified to create the other classes of BBSR polypeptides. For example, mutants can be created by making conservative amino acid substitutions that maintain or enhance native BBSR protein or the protein's activity. The following are examples of conservative substitutions: Gly [symbol] Ala; Val [symbol] Ile [symbol] Leu; Asp [symbol] Glu; Lys [symbol] Arg; Asn [symbol] Gin; and Phe [symbol] Trp [symbol] Tyr. Mutants can also contain amino acid deletions or insertions compared to the native BBSR polypeptides. Mutants may include substitutions, insertions, and deletions of the native polypeptides.

Mutants will retain at least about 20% of the one of the activities of the native BBSR protein. The coding sequence of mutants can be constructed by in vitro mutagenesis of the native coding sequences.

Fragments differ from mutant or native BBSR polypeptides by amino and/or carboxyl terminal amino acid deletions. The number of amino acids that are truncated is not critical as long as the BBSR protein fragment retains at least about 20% of the one of the activities of the native BBSR polypeptide. The coding sequence of such fragments can be easily constructed by cleaving the unwanted nucleotides from the mutant or native BBSR polypeptide coding sequences.

Fusions are fragments, mutants, or native BBSR polypeptides with additional amino acids at either or both of the termini. The additional amino acid sequence is not necessarily homologous to sequence found in native polypeptides. The fusions, just as all BBSR polypeptides, retain at least about 20% of one of the activities of the native BBSR polypeptides. Coding sequence of the fusions can be constructed by ligating synthetic polynucleotides encoding the additional amino acids to fragment, mutant, or native coding sequences. Activities of the BBSR polypeptides can be determined by the methods described infra.

At the minimum, an expression vector will contain a promoter which is operable, that is drives expression in the host cell and operably linked to a BBSR protein or polypeptide coding sequence. Sequences that modulate gene expression, such as enhancers and binding sites for inducers or repressors may be present. Expression vectors may also include signal sequences, terminators, selectable markers, origins of replication, and sequences homologous to host cell sequences. These additional elements are optional but can be included to optimize expression. Construction of expression vectors is known in the art and any appropriate methods can be employed with the polynucleotides of the invention.

A BBSR protein or polypeptide coding sequence may also be linked in reading frame to a signal sequence. The signal sequence fragment typically encodes a peptide comprised of hydrophobic amino acids which directs the BBSR protein or polypeptide to the cell membrane or other subcellular compartment. Preferably, there are processing sites encoded between the leader fragment and the gene or fragment thereof that can be cleaved either in vivo or in vitro. DNA encoding suitable signal sequences can be derived from genes for secreted endogenous host cell proteins, such as the yeast invertase gene (EP 12 873, JP 62,096,086), the A-factor gene (U.S. Pat. No. 4,588,684), interferon signal sequence (EP 60 057).

After vector construction, the desired BBSR protein and/or BBSR polypeptide expression vector is inserted into the host cell. Many transformation techniques exist for inserting expression vectors into bacterial, yeast, insect, and mammalian cells. The transformation procedure to introduce the expression vector depends upon the host to be transformed. Such methods are known in the art. See, e.g., (Masson et al. (1989) FEMS Microbiol. Lett. 60:273; Palva et al. (1982) Proc. Natl. Acad. Sci. USA 79:5582; EP Publ. Nos. 036 259 and 063 953; PCT WO 84/04541, Bacillus), (Miller et al. (1988) Proc. Natl. Acad. Sci. 85:856; Wang et al. (1990) J. Bacteriol. 172.949, Campylobacter), (Cohen et al. (1973) Proc. Natl. Acad. Sci. 69:2110; Dower et al. (1988) Nuc. Acids Res. 16:6127; Kushner et al. (1978) “An Improved Method for Transformation of Escherichia coli with ColE1-derived plasmids in Genetic Engineering: Proceedings of the International Symposium on Genetic Engineering (eds. H. W. Boyer and S. Nicosia); Mandel et al. (1970) J. Mol. Biol. 53:159; Taketo et al. (1988) Biochim. Biophys. Acta 949:318; Escherichia), (Chassy et al. (1987) FEMS Microbiol. Lett. 44:173, Lactobacillus); (Fiedler et al. (1988) Anal. Biochem. 170:38, Pseudomonas); (Augustin et al. (1990) FEMS Microbiol. Lett. 66:203, Staphylococcus), Barany et al. (1980) J. Bacteriol. 144:698; Harlander et al. (1987) “Transformation of Streptococcus lactis by electroporation,” in Streptococcal Genetics (ed. J. Ferretti and R. Curtiss, III); Perry et al. (1981) Infec. Immun. 32:1295; Powell et al. (1988) Appl. Environ. Microbiol. 54:655; Somkuti et al. (1987) Proc. 4th Evr. Cong. Biotechnology 1:412, Streptococcus).

Transformation methods for yeast hosts are well-known in the art, and typically include either the transformation of spheroplasts or of intact yeast cells treated with alkali cations. Electroporation is another means for transforming yeast hosts. See, for example, Methods in Enzymology, Volume 194, 1991, “Guide to Yeast Genetics and Molecular Biology” Transformation procedures usually vary with the yeast species to be transformed. See, e.g., (Kurtz et al (1986) Mol. Cell. Biol. 6:142; Kunze et al. (1985) J. Basic Microbiol. 25:141, Candida); (Gleeson et al. (1986) J. Gen. Microbiol. 132:3459; Roggenkamp et al. (1986) Mol. Gen. Genet. 202:302, Hansemila); (Das et al (1984) J. Bacteriol. 158:1165; De Louvencourt et al. (1983) J. Bacteriol. 154:1165; Van den Berg et al. (1990) Biotechnology 8:135, Kluyveromyces); (Cregg et al. (1985) Mol. Cell. Biol. 5:3376; Kunze et al. (1985) J. Basic Microbiol. 25:141; U.S. Pat. Nos. 4,837,148 and 4,929,955, Pichia); (Hinnen et al. (1978) Proc. Natl. Acad. Sci. USA 75:1929; Ito et al. (1983) J. Bacteriol. 153:163, Saccharomyces); (Beach and Nurse (1981) Nature 300:706, Schizosaccharomyces); (Davidow et al. (1985) Curr. Genet. 10:39; Gaillardin et al. (1985) Curr. Genet. 10:49, Yarrowia).

Methods for introducing heterologous polynucleotides into mammalian cells are known in the art and include viral infection, dextran-mediated transfection, calcium phosphate precipitation, microparticle bombardment, protoplast fusion, electroporation, encapsulation of the polynucleotide(s) in liposomes, and direct microinjection of the DNA into nuclei.

Monitoring BBSR Polypeptide Expression Levels

Immunoassays and ligand binding assays can be utilized to confirm that the transformed host cell is expressing the desired BBSR polypeptide Polyclonal or monoclonal antibodies to BBSR proteins can be prepared by any methods known in the art, using as immunogen the whole BBSR protein or an epitope-bearing portion thereof, which can comprise between about 10 and 100 contiguous amino acids of SEQ ID NO:2, SEQ ID NO:4, SEQ ID NO:6, or SEQ ID NO:9, preferably between about 12 and 50 contiguous amino acids of SEQ ID NO:2, SEQ ID NO:4, SEQ ID NO:6, or SEQ ID NO:9, more preferably between about 15 and 25 contiguous amino acids of SEQ ID NO:2, SEQ ID NO:4, SEQ ID NO:6, or SEQ ID NO:9.

For example, an immunofluorescence assay can be performed on transformed host cells without separating the BBSR polypeptides from the cell. The host cells are first fixed onto a solid support, such as a microscope slide or microtiter well. This fixing step permeabilizes the cell membrane. Next, the fixed host cells are exposed to an anti-BBSR polypeptide antibody Preferably, to increase the sensitivity of the assay, the fixed cells are exposed to a second antibody, which is labeled and binds to the anti-BBSR polypeptide antibody. Typically, the secondary antibody is labeled with a fluorescent marker. The host cells, which express the BBSR polypeptides, will be fluorescently labeled and easily visualized under the microscope. See, for example, Hashido et al. (1992) Biochem & Biophys. Res. Comm. 187(3):1241-1248.

Also, the BBSR polypeptides do not need to be separated from the cell membrane for in vitro assays. The host cells may be fixed to a solid support, such as a microtiter plate. Alternatively, a crude membrane fraction can be separated from lysed host cells by centrifugation (See Adachi et al. (1992) FEBS Lett 311(2):179-183. The fixed host cells or the crude membrane fraction is exposed to labeled ligand or ion. Typically, the ligand is labeled with radioactive atoms. The host cells, which express the desired BBSR polypeptide, will bind with the labeled ligand, which can be easily detected.

BBSR polypeptides can be purified and are useful as compositions, for assays, and to produce antibodies. BBSR polypeptides can be isolated by a variety of steps including, for example, anion exchange chromatography, size exclusion chromatography, hydroxylapatite chromatography, hydrophobic interaction chromatography, metal chelation chromatography, reverse phase HPLC, affinity chromatography, and further ammonium sulfate precipitations. These techniques are well known to those of skill in the art.

For ligand binding studies, patch clamp analysis or other in vitro assays, the crude cell membrane fractions can be utilized. These membrane extracts can be isolated from cells, which expressed BBSR polypeptides by lysing the cells. Alternatively, whole cells, expressing BBSR polypeptides, can be cultured in a microtiter plate.

Antibodies

Antibodies against BBSR polypeptides are useful for affinity chromatography, immunofluorescent assays, and distinguishing BBSR polypeptides; and for inhibiting or modulating an activity or biological effect or a disorder associated with the BBSR-proteins of the invention Such uses include but are not limited to modulation of BBSR-mediated disorders; modulation of transcription, particularly that of apo A1, modulation of ion transport, cholesterol homeostasis, and the like.

Antibodies to the proteins of the invention, both polyclonal and monoclonal, may be prepared by conventional methods known to those skilled in the art. For example, monoclonal antibodies are prepared using the method of Kohler et al. (1975) Nature 256:495-496, or a modification thereof If desired, the antibodies (whether polyclonal or monoclonal) may be labeled using conventional techniques. Suitable labels include fluorophores, chromophores, radioactive atoms (particularly ³²P and ¹²⁵I), electron-dense reagents, enzymes, and ligands having specific binding partners. Enzymes are typically detected by their activity. For example, horseradish peroxidase is usually detected by its ability to convert 3,3′,5,5′-tetra-methylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, ¹²⁵I may serve as a radioactive label or as an electron-dense reagent. HRP may serve as enzyme or as antigen for a MAb. Further, one may combine various labels for desired effect. For example, MAbs and avidin also require labels in the practice of this invention: thus, one might label a MAb with biotin, and detect its presence with avidin labeled with ¹²⁵I, or with an anti-biotin MAb labeled with HRP. Other permutations and possibilities will be readily apparent to those of ordinary skill in the art, and are considered as equivalents within the scope of the instant invention.

Ion Transport Activity

The channel forming and ion transport activity of the plasmolipin-like polypeptide (SEQ ID NO:4) is determined essentially as described in U.S. Pat. No. 5,843,714. The plasmolipin-like polypeptide is assayed by monitoring its effect on transmembrane pH gradients in liposomes. Mitochondrial cytochrome C oxidase, a proton pump, is reconstituted into liposomes by sonication. The pH-sensitive fluorescent dye pyranine (Eastman Kodak) is then incorporated into the proteoliposomes by rapid freeze-thawing and sonication. Excess dye is removed by centrifugation and resuspension of the liposomes into an appropriate buffer. Addition of ascorbate and cytochrome C initiates proton uptake into the liposomes. PLP protein is added and proton efflux is monitored by the fluorescence changes arising from changes in internal pH of the liposomes at excitation and emission wavelengths of 460 nm and 508 nm, respectively.

Lipid bilayer destabilization promoted by the plasmolipin-like polypeptide, incorporated into membranes by expression or by reconstitution, is assayed by measurement of the fluorescence polarization of the lipophilic dye 1,6-diphenyl-1,3,5-hexatriene (Eastman Kodak) inserted into the membranes.

Screening for Agonists and Antagonists

BBSR polypeptides can also be used to screen combinatorial libraries to identify agonist or antagonists. For example, a “library” of peptides may be synthesized following the methods disclosed in U.S. Pat. No. 5,010,175, and in PCT WO 91/17823, both incorporated herein by reference in full. The peptide library is first screened for binding to the selected BBSR polypeptide. The peptides are then tested for their ability to inhibit or enhance the particular BBSR protein activity. Peptides exhibiting the desired activity are then isolated and sequenced.

Agonists or antagonists of BBSR proteins may be screened using any available method. The assay conditions ideally should resemble the conditions under which the activity of the particular BBSR protein is exhibited in vivo, i.e., under physiologic pH, temperature, ionic strength, etc. Suitable agonists or antagonists will exhibit strong inhibition or enhancement of the particular activity of the BBSR protein at concentrations, which do not raise toxic side effects in the subject. Agonists or antagonists which compete for binding to the BBSR polypeptide may require concentrations equal to or greater than the native BBSR protein concentration, while inhibitors capable of binding irreversibly to the polypeptide may be added in concentrations on the order of the native BBSR protein concentration.

Pharmaceutical Compositions

Pharmaceutical compositions can comprise either polypeptides, antibodies, or polynucleotides of the claimed invention. The pharmaceutical compositions will comprise a therapeutically effective amount of either polypeptides, antibodies, or polynucleotides of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgment of the clinician.

For purposes of the present invention, an effective dose will be from about 0.01 mg/kg to 50 mg/kg or 0.05 mg/kg to about 10 mg/kg of the polypeptide or DNA construct in the individual to which it is administered

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (1991) (Mack Pub. Co., NJ).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

Delivery Methods

Once formulated, the compositions of the invention can be administered directly to the subject. The subjects to be treated can be mammals or birds. In particular, human subjects can be treated

Direct delivery of the compositions will generally be accomplished by injection, either subcutaneously, intraperitoneally, intravenously or intramuscularly or delivered to the interstitial space of a tissue. The compositions can also be administered into a tumor or lesion. Other modes of administration include oral and pulmonary administration, suppositories, and transdermal applications, needles, and gene guns or hyposprays. Dosage treatment may be a single dose schedule or a multiple dose schedule.

Alternatively, the BBSR polypeptides could be stably expressed in an organ of a mammal, and then the organ could be xenografted into a human in need of such treatment.

Gene Delivery Vehicles

Gene therapy vehicles for delivery of constructs including a coding sequence of a therapeutic of the invention, to be delivered to the mammal for expression in the mammal, can be administered either locally or systemically. These constructs can utilize viral or non-viral vector approaches in in vivo or ex vivo modality. Expression of such coding sequence can be induced using endogenous mammalian or heterologous promoters. Expression of the coding sequence in vivo can be either constitutive or regulated.

The invention includes gene delivery vehicles capable of expressing the contemplated nucleic acid sequences. The gene delivery vehicle is preferably a viral vector and, more preferably, a retroviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, or togavirus viral vector. See generally, Jolly et al. (1994) Cancer Gene Therapy 1:51-64; Kimura et al. (1994) Human Gene Therapy 5:845-852; Connelly et al. (1995) Human Gene Therapy 6:185-193; and Kaplitt et al. (1994) Nature Genetics 6.148-153.

Retroviral vectors are well known in the art and it is contemplated that any retroviral gene therapy vector is employable in the invention, including B, C and D type retroviruses, xenotropic retroviruses (for example, NZB-X1, NZB-X2 and NZB9-1 (see O'Neill (1985) J. Vir. 53:160) polytropic retroviruses (for example, MCF and MCF-MLV (see Kelly et al. (1983)J. Vir. 45:291), spumaviruses and lentiviruses. See RNA Tumor Viruses, Second Edition, Cold Spring Harbor Laboratory, 1985.

Portions of the retroviral gene therapy vector may be derived from different retroviruses. For example, retrovector LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

These recombinant retroviral vectors may be used to generate transduction competent retroviral vector particles by introducing them into appropriate packaging cell lines (see U.S. Ser. No. 07/800,921, filed Nov. 29, 1991). Retrovirus vectors can be constructed for site-specific integration into host cell DNA by incorporation of a chimeric integrase enzyme into the retroviral particle. See, U.S. Ser. No. 08/445,466 filed May 22, 1995. It is preferable but not required that the recombinant viral vector is a replication defective recombinant virus.

Packaging cell lines suitable for use with the above-described retrovirus vectors are well known in the art, are readily prepared (see U.S. Ser. No. 08/240,030, filed May 9, 1994; see also WO 92/05266), and can be used to create producer cell lines (also termed vector cell lines or “VCLs”) for the production of recombinant vector particles. Preferably, the packaging cell lines are made from human parent cells (e.g., HT1080 cells) or mink parent cell lines, which eliminates inactivation in human serum.

Preferred retroviruses for the construction of retroviral gene therapy vectors include Avian Leukosis Virus, Bovine Leukemia, Virus, Murine Leukemia Virus, Mink-Cell Focus-Inducing Virus, Murine Sarcoma Virus, Reticuloendotheliosis Virus and Rous Sarcoma Virus. Particularly preferred Murine Leukemia Viruses include 4070A and 1504A (Hartley et al. (1976) J. Virol. 19:19-25), Abelson (ATCC No. VR-999), Friend (ATCC No. VR-245), Graffi, Gross (ATCC No. VR-590), Kirsten, Harvey Sarcoma Virus and Rauscher (ATCC No. VR-998) and Moloney Murine Leukemia Virus (ATCC No. VR-190). Such retroviruses may be obtained from depositories or collections such as the American Type Culture Collection (“ATCC”) in Rockville, Md. or isolated from known sources using commonly available techniques.

The gene delivery vehicles of the invention also include adenovirus associated virus (AAV) vectors. Leading and preferred examples of such vectors for use in this invention are the AAV-2 based vectors disclosed in Srivastava, WO 93/09239.

Also contemplated are alpha virus gene therapy vectors that can be employed in this invention. Preferred alpha virus vectors are Sindbis viruses vectors. Togaviruses, Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described in U.S. Pat. Nos. 5,091,309, 5,217,879, and WO 92/10578, WO 95/07994, U.S. Pat. No. 5,091,309 and U.S. Pat. No. 5,217,879 are employable. Such alpha viruses may be obtained from depositories or collections such as the ATCC in Rockville, Md. or isolated from known sources using commonly available techniques. Preferably, alphavirus vectors with reduced cytotoxicity are used (see co-owned U.S. Ser. No. 08/679,640).

### EXAMPLES

The example presented below is provided as a further guide to the practitioner of ordinary skill in the art, and is not to be construed as limiting the invention in any way.

### Example 1

### Polynucleotides that Map to Regions of the Human Genome Associated With BBS

A 2850 bp cDNA (SEQ ID NO:1) from human brain (frontal cortex) that encodes an open reading frame of 762 bp (encompassing bases 214-976) was isolated and is referred to herein as Gene X. The Gene X gene is located on human chromosome 16. Northern analysis showed that the transcript of Gene X is expressed in brain (cerebellum, cerebral cortex, medulla, spinal cord, occipital lobe, frontal lobe, temporal lobe and putamen), heart, skeletal muscle, spleen, testis, and pancreas, with very low expression in thymus, ovary, small intestine, colon, peripheral blood leukocytes, placenta, lung liver and kidney. The transcript corresponds to a minimum of 4 exons.

A 1516 bp cDNA (SEQ ID NO:3) was isolated from human brain that encodes an open reading frame of 545 bp (encompassing bases 210-755 bp nucleotide). This gene is located on human chromosome 16. The nucleotide sequence shares homology with rat plasmolipin. At the amino acid level it has about 89% identity with rat plasmolipin and with NTII-11. The transcript of this novel gene is expressed in brain, kidney, lung, pancreas, spleen, prostate, heart, testis, small intestine, liver, colon, skeletal muscle, placenta and ovary, but is not visible on Northerns of thymus and peripheral blood leukocytes.

A 2177 bp cDNA (SEQ ID NO:5) was isolated from human retina that encodes an open reading frame of 891 bp (encompassing bases 167-1057). This gene is located on human chromosome 15. At the nucleotide level it contains over 70% identity with several transcription factors (e.g., tailless, chick ovalbumin upstream promoter transcription factor II, apolipoprotein A1 regulatory protein) over regions spanning 224 bp. The transcript of this novel gene is about 1.5 kb on Northern and was expressed in brain (cerebellum, cerebral cortex, medulla, spinal cord, occipital lobe, frontal lobe, temporal lobe and putamen), heart, placenta, lung, liver, skeletal muscle, kidney, pancreas, spleen, thymus, prostate, testis, ovary, small intestine, colon, and peripheral blood leukocytes. The transcript contains an open reading frame of 891 bp encompassing bases 167-1057 and predicts a protein of 297 amino acids.

Two fragments of 214 bp (SEQ ID NO:7) and 65 bp were identified which are located on BAC 17354 (from human chromosome 16). A 126 bp fragment was isolated for use as a probe for hybridization on Northern blots and this probe identified a transcript of 2.3 kb that was present in brain (cerebellum, cerebral cortex, medulla, spinal cord, occipital lobe, frontal lobe, temporal lobe and putamen), heart, placenta, lung, liver skeletal muscle, kidney, pancreas, spleen, prostate, ovary, small intestine, colon, and peripheral blood leukocytes. A slightly larger transcript, ˜2.4 kb, was present in testis and no expression appeared in thymus RNA

Identities were determined using MPSRCH™ software, Release 3.3A (distributed by Oxford Molecular Ltd.). Nucleotide identity determinations were made using MPSRCH_nn, using Smith-Waterman algorithm using default TABLE Gap 6.

Protein identity determinations were made either using MPsrch-PP, using Smith-Waterman algorithm, using blosum 60 TABLE, Gap 14; or by using blosum 60 TABLE, Base Gap open 24, Gap extend 3, A-A Gap open 30, Gap extend 9.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. An isolated nucleic acid molecule comprising a polynucleotide selected from the group consisting of:
(a) a polynucleotide encoding amino acids from about 1 to about 254 of SEQ ID NO:2;
(b) a polynucleotide encoding amino acids from about 2 to about 254 of SEQ ID NO:2;
(c) a polynucleotide encoding amino acids from about 1 to about 218 of SEQ ID NO:4;
(d) a polynucleotide encoding amino acids from about 2 to about 218 of SEQ ID NO:4;
(e) a polynucleotide encoding amino acids from about 1 to about 297 of SEQ ID NO:6;
(f) a polynucleotide encoding amino acids from about 2 to about 297 of SEQ ID NO:6;
(g) a polynucleotide encoding amino acids from about 1 to about 513 of SEQ ID NO:9;
(h) a polynucleotide encoding amino acids from about 2 to about 513 of SEQ ID NO:9;
(i) a polynucleotide consisting of SEQ ID NO:7;
(j) the polynucleotide complement of the polynucleotide of any one of (a) through (i); and
(k) a polynucleotide at least 90% identical to the polynucleotide of any one of (a) through (j).

2. An isolated nucleic acid molecule comprising about 762 contiguous nucleotides from the coding region of SEQ ID NO:1, about 545 contiguous nucleotides from the coding region of SEQ ID NO:3, about 891 contiguous nucleotides from the coding region of SEQ ID NO:5, or about 1539 contiguous nucleotides from the coding region of SEQ ID NO:8.

3. An isolated nucleic acid molecule comprising a polynucleotide encoding a polypeptide wherein, except for at least one conservative amino acid substitution, said polypeptide has an amino acid sequence selected from the group consisting of:
(a) amino acids from about 1 to about 254 of SEQ ID NO:2;
(b) amino acids from about 2 to about 254 of SEQ ID NO:2;
(c) amino acids from about 1 to about 218 of SEQ ID NO:4;
(d) amino acids from about 2 to about 218 of SEQ ID NO:4;
(e) amino acids from about 1 to about 297 of SEQ ID NO:6;
(f) amino acids from about 2 to about 297 of SEQ ID NO:6;
(g) amino acids from about 1 to about 513 of SEQ ID NO:9;
(h) amino acids from about 2 to about 513 of SEQ ID NO:9.

4. The isolated nucleic acid molecule of claim 1, which is DNA.

5. A method of making a recombinant vector comprising inserting a nucleic acid molecule of claim 1 into a vector in operable linkage to a promoter.

6. A recombinant vector produced by the method of claim 5.

7. A method of making a recombinant host cell comprising introducing the recombinant vector of claim 6 into a host cell.

8. A recombinant host cell produced by the method of claim 7.

9. A recombinant method of producing a polypeptide, comprising culturing the recombinant host cell of claim 8 under conditions such that said polypeptide is expressed and recovering said polypeptide.

10. An isolated polypeptide comprising amino acids at least 95% identical to amino acids selected from the group consisting of:
(a) amino acids from about 1 to about 254 of SEQ ID NO:2;
(b) amino acids from about 2 to about 254 of SEQ ID NO:2;
(c) amino acids from about 1 to about 218 of SEQ ID NO:4;
(d) amino acids from about 2 to about 218 of SEQ ID NO:4;
(e) amino acids from about 1 to about 297 of SEQ ID NO:6;
(f) amino acids from about 2 to about 297 of SEQ ID NO:6;
(g) amino acids from about 1 to about 513 of SEQ ID NO:9;
(h) amino acids from about 2 to about 513 of SEQ ID NO:9.

11. An isolated polypeptide wherein, except for at least one conservative amino acid substitution, said polypeptide has an amino acid sequence selected from the group consisting of:
(a) amino acids from about 1 to about 254 of SEQ ID NO:2;
(b) amino acids from about 2 to about 254 of SEQ ID NO:2;
(c) amino acids from about 1 to about 218 of SEQ ID NO:4;
(d) amino acids from about 2 to about 218 of SEQ ID NO:4;
(e) amino acids from about 1 to about 297 of SEQ ID NO:6;
(f) amino acids from about 2 to about 297 of SEQ ID NO:6;
(g) amino acids from about 1 to about 513 of SEQ ID NO:9;
(h) amino acids from about 2 to about 513 of SEQ ID NO:9.

12. An isolated polypeptide comprising amino selected from the group consisting of:
(a) amino acids from about 1 to about 254 of SEQ ID NO:2;
(b) amino acids from about 2 to about 254 of SEQ ID NO:2;
(c) amino acids from about 1 to about 218 of SEQ ID NO:4;
(d) amino acids from about 2 to about 218 of SEQ ID NO:4;
(e) amino acids from about 1 to about 297 of SEQ ID NO:6;
(f) amino acids from about 2 to about 297 of SEQ ID NO:6;
(g) amino acids from about 1 to about 513 of SEQ ID NO:9;
(h) amino acids from about 2 to about 513 of SEQ ID NO:9.

13. An epitope-bearing portion of a polypeptide selected from the group consisting of SEQ ID NO:2, SEQ ID NO:4, SEQ ID NO:6, and SEQ ID NO:9.

14. The epitope-bearing portion of claim 13, which comprises between about 10 and 100 contiguous amino acids of SEQ ID NO:2, SEQ ID NO:4, SEQ ID NO:6, or SEQ ID NO:9.

15. The epitope-bearing portion of claim 14, which comprises between about 12 and 50 contiguous amino acids of SEQ ID NO:2, SEQ ID NO:4, SEQ ID NO:6, or SEQ ID NO:9.

16. The epitope-bearing portion of claim 14, which comprises between about 15 and 25 contiguous amino acids of SEQ ID NO:2, SEQ ID NO:4, SEQ ID NO:6, or SEQ ID NO:9.

17. An isolated antibody that binds specifically to the polypeptide of claim 10.

18. An isolated antibody that binds specifically to the polypeptide of claim 11.

19. An isolated antibody that binds specifically to the polypeptide of claim 12.

20. A method for diagnosing a BBSR protein-modulated disorder using a biological sample from a human suspected of having said disorder, said method comprising:
a) providing an antibody that binds to the polypeptide of claim 10;
b) contacting the antibody with said sample under binding conditions to form a duplex; and
c) determining the amount of said duplex formed, compared to a normal sample.

21. A method for diagnosing a BBSR protein-modulated disorder in a biological sample from a human suspected of having said disorder, said method comprising:
a) providing a polynucleotide that binds to mRNA encoding the polypeptide of claim 10 under stringent conditions;
b) contacting nucleic acid of said sample with said polynucleotide under binding conditions to form a duplex; and
c) determining the amount of said duplex formed, compared to a normal sample.

22. A method for modulating the amount of a BBSR protein in a subject, said method comprising administering an effective amount of a composition selected from a group consisting of:
a) the polypeptide according to claim 10; and
b) an antibody that binds to the polypeptide according to claim 10.

23. A method for modulating the amount of a BBSR protein in a subject, said method comprising administering an effective amount of a composition consisting of the nucleotide sequence according to claim 1.

24. A method for treating a BBSR protein-modulated disorder in a subject, said method comprising administering to said subject an effective amount of a composition selected from a group consisting of:
a) the polypeptide according to claim 10; and
b) an antibody that binds to the polypeptide according to claim 10; wherein said composition further comprises a pharmaceutically acceptable carrier.

25. A method for treating a BBSR protein-modulated disorder in a subject, said method comprising administering to said subject an effective amount of a composition consisting of the nucleotide sequence according to claim 1, wherein said composition further comprises a pharmaceutically acceptable carrier.
